(19) United States
(12) Patent Application Publication
(10) Pub. No.: US 2005/0102711 A1
(43) Pub. Date: May 12, 2005

(54) Banana actin gene and its promoter

(76) Inventors: James Langham Dale, Queensland (AU); Robert Maxwell Harding, Queensland (AU); Scott Richard Hermann, Queensland (AU)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/451,889

(22) PCT Filed: Dec. 28, 2001
(86) PCT No.: PCT/AU01/01679

(30) Foreign Application Priority Data
Dec. 28, 2000 (AU) PR 2328

(51) Int. Cl. C12Q 1/68; A01H 1/00; C12N 15/82

(52) U.S. Cl. 800/278

(57) ABSTRACT

The present invention is directed to the isolation and identification of a non-graminaceous monocotyledonous plant promoter, the banana (Musa.Spp.) actinn gene associated promoter region. The promoter region has been found to unexpectedly direct constitutive gene expression not only in non-graminaceous monocotyledonous plants but also in graminaceous monocotydonous plants. The Invention is also concerned with a chimeric nucleic acid construct comprising the promoter of the invention oprably linked to a foreign or endogenous polynucleotide encoding a protein of interest or a transcript capable of modulating expression of a target gene. The invention further discloses transformed plant cells, as well as differentiated plants and plant parts, containing the construct.

### FIELD OF THE INVENTION

THIS INVENTION relates generally to promoters for use in plant genetic engineering. More particularly, the present invention relates to a constitutive promoter for expression of foreign or endogenous coding sequences in plants, including monocotyledonous plants. The invention is also concerned with a chimeric nucleic acid construct comprising the promoter of the invention operably linked to a foreign or endogenous polynucleotide encoding a protein of interest or a transcript capable of modulating expression of a target gene. The invention is further concerned with transformed plant cells, as well as differentiated plants and plant parts, containing the construct.

Bibliographic details of various publications referred to by author in this specification are collected at the end of the description.

### BACKGROUND OF THE INVENTION

A primary goal of genetic engineering is to obtain plants having improved characteristics or traits. Many different types of characteristics or traits are considered advantageous, but those of particular importance include resistance to plant diseases, resistance to viruses or insects and resistance to herbicides. Other advantageous characteristics or traits include tolerance to cold or soil salinity, enhanced stability or shelf life of the ultimate consumer product obtained from a plant, or improvement in the nutritional value of edible portions of a plant.

Recent advances in genetic engineering have enabled the incorporation of a selected gene (or genes) into plant cells to impart a desired quality (or qualities) to a plant of interest. The selected gene (or genes) may be derived from a source different from the plant of interest or may be native to the desired plant, but engineered to have different or improved qualities. This new gene (or genes) may then be expressed in cells of the regenerated plant to exhibit the new trait or characteristic.

In order for the newly incorporated gene to express the protein for which it codes in a plant cell, the proper regulatory signals must be present and in the proper location with respect to the gene. These regulatory signals include a promoter, a 5′ non-translated leader sequence and a 3′ polyadenylation signal.

The efficiency of gene expression is governed largely by the promoter used to express the gene. A promoter is typically a DNA sequence that directs the cellular machinery of a plant to produce (transcribe) RNA (transcript) from a contiguous transcribable region downstream (3′) of the promoter. The promoter influences the rate at which the transcript of the gene is made. Assuming the transcript includes a coding region with appropriate translational signals, the promoter also influences the rate at which the resultant protein product of the gene is produced. Promoter activity also can depend on the presence of several other cis-acting regulatory elements which, in conjunction with cellular factors, determine strength, specificity, and transcription initiation site (for a review, see Zawel and Reinberg, 1992, Curr. Opin. Cell Biol. 4: 488).

It has been shown that certain promoters are able to direct RNA synthesis at a higher rate relative to other promoters. These are called “strong promoters”. Certain other promoters have been shown to direct RNA production at higher levels only in particular types of cells or tissues and are often referred to as “tissue-specific promoters”. Promoters that are capable of directing RNA production in many or all tissues of a plant are called “constitutive promoters”. Thus, expression of a chimeric gene (or genes) introduced into a plant may potentially be controlled by identifying and using a promoter with the desired characteristics.

There have been numerous promoters described for gene expression in dicotyledonous plants. However, there still remains a dearth of promoters that can be used for effective expression of foreign or endogenous coding sequences in monocotyledonous plants.

One promoter that has been used widely for directing gene expression in graminaceous monocotyledonous plants is the rice actin (ACT1) promoter (McElroy et al., 1990, Plant Cell 2: 163-171; Takumi et al., 1994, Plant Science 103: 161-66; Zhong et al., 1996, Plant Science 116: 73-84; Chibbar et al., 1993, Plant Cell Rep 12: 506-509). However, its expression in certain non-graminaceous monocots is variable (Wilmink et al., 1995, Plant Mol Biol 28: 949-955) suggesting that the activity of actin promoters may be limited to closely related species.

### SUMMARY OF THE INVENTION

The present invention is predicated in part on the discovery of an actin gene from a non-graminaceous monocotyledonous plant and its associated promoter region. The promoter region, in particular, has been found quite unexpectedly to direct constitutive gene expression not only in non-graminaceous monocotyledonous plants but also in graminaceous monocotyledonous plants. The foregoing discovery has been reduced to practice in novel isolated DNA molecules, promoter regions, chimeric DNA constructs as well as plant cells and differentiated plants containing them, as described hereinafter.

Thus, in one aspect, the invention provides an isolated DNA molecule comprising a promoter or biologically active fragment thereof or variant of these, wherein said promoter is located upstream of a transcribable DNA sequence that hybridises to a nucleic acid probe derived from the polynucleotide sequence set forth in SEQ ID NO: 1.

Advantageously, the isolated promoter is of sufficient length such that it is capable of initiating and regulating transcription of a DNA sequence to which it is coupled. The promoter may be between 100 bp and 4 kb in length and preferably greater than 1 kb in length. An analogous promoter can be obtained from any plant species that has a DNA sequence that is transcribed in one or more cells or tissues of the plant provided that the DNA sequence is capable of hybridising to a nucleic acid probe derived from the polynucleotide sequence as set forth in SEQ ID NO: 1 under at least low stringency conditions, preferably under at least medium stringency conditions, and more preferably under high stringency conditions.

The polynucleotide sequence set forth in SEQ ID NO: 1 is a transcribable sequence obtained from banana (Musa spp.). The polynucleotide sequence identified by SEQ ID NO: 1 is expressed constitutively in leaves, stems, roots and flowers of banana. Homologous sequences corresponding to this polynucleotide are predicted to be expressed in many or all tissues of other monocotyledonous plants including non-graminaceous monocotyledonous plants such as taro, ginger, onions, garlic, pineapple, bromeliaeds, palms, orchids, lilies, irises and the like.

Accordingly, the invention also features a polynucleotide sequence or variant thereof that is highly transcribed constitutively in tissue of monocotyledonous plants wherein said sequence comprises the polynucleotide sequence set forth in SEQ ID NO: 1.

The foregoing polynucleotide sequence represents a transcribable sequence, which can be used to make a probe for isolating homologous transcribable sequences in other plant species, preferably, monocotyledonous species, and more preferably non-graminaceous monocotyledonous species, so that a corresponding promoter from said other plant species having the same constitutive qualities can be isolated and used.

Preferably, the promoter comprises the sequence set forth in SEQ ID NO: 3.

Suitably, the biologically active fragment is selected from any tone of the sequences set forth in SEQ ID NO: 4, 5, 6 and 7.

In one embodiment, the variant has at least 60%, preferably at least 80%, more preferably at least 90%, and most preferably at least 95% sequence identity to any one of the polynucleotides identified by SEQ ID NO: 3, 4, 5, 6 and 7.

In another embodiment, the variant is capable of hybridising to any one of the polynucleotides identified by SEQ ID NO: 3, 4, 5, 6 and 7 under at least low stringency conditions, preferably under at least medium stringency conditions, and more preferably under high stringency conditions.

Suitably, a promoter according to the invention can be fused to a desired coding sequence to create a chimeric construct. This construct can then be introduced into a host cell, preferably a plant cell or plant or plant part, by any method of choice.

Accordingly, in another aspect of the invention, there is provided a chimeric DNA construct comprising an isolated promoter or biologically active fragment thereof or variant of these, wherein said promoter is naturally located upstream of a transcribable DNA sequence which hybridises to a nucleic acid probe derived from the polynucleotide sequence set forth in SEQ ID NO: 1, wherein said promoter or biologically active fragment or variant is operably linked to a foreign or endogenous DNA sequence to be transcribed.

Suitably, the chimeric DNA construct further comprises a 3′ non-translated sequence that is operably linked to the foreign or endogenous DNA sequence and that functions in plant cells to terminate transcription and/or to cause addition of a polyadenylated nucleotide sequence to the 3′ end of a transcribed RNA sequence.

The foreign or endogenous DNA sequence is foreign or endogenous with respect to the plant cell in which it is or will be introduced. In one embodiment, the foreign or endogenous DNA sequence encodes a structural or regulatory protein. In an alternate embodiment, the foreign or endogenous DNA sequence encodes a transcript capable of modulating expression of a corresponding target gene.

In a preferred embodiment, the transcript comprises an antisense RNA or a ribozyme or other transcribed region aimed at downregulation of expression of the corresponding target gene. For example, the said other transcribed region may comprise a sense transcript aimed at sense suppression (co-suppression) of the corresponding target gene.

Depending on the polynucleotide selected, in one embodiment, the chimeric DNA construct may be further characterised in that said promoter or biologically active fragment or variant is capable of conferring transcription, preferably high levels of transcription, of the foreign or endogenous DNA sequence in many or all tissues of a plant.

In a further aspect, the invention provides a method for gene expression in a plant, comprising introducing into a plant cell a chimeric DNA construct as broadly described above.

In still yet another aspect, the invention contemplates a method for producing transformed plant cells, comprising introducing into regenerable plant cells a chimeric DNA construct as broadly described above so as to yield transformed plant cells and identifying or selecting transformed plant cells.

In yet another aspect, the invention provides a method for selecting stable genetic transformants from transformed plant cells, comprising introducing into regenerable plant cells a chimeric DNA construct as broadly described above so as to yield transformed plant cells and identifying or selecting a transformed plant cell line from said transformed plant cells.

In a preferred embodiment, the regenerable cells are regenerable dicotyledonous plant cells, preferably monocotyledonous plant cells, more preferably regenerable graminaceous monocotyledonous plant cells and even more preferably regenerable non-graminaceous monocotyledonous plant cells. In another preferred embodiment, the expression of the chimeric DNA construct in the transformed cells imparts a phenotypic characteristic to the transformed cells.

According to another aspect of the invention, there is provided a transformed plant cell containing a chimeric DNA construct as broadly described above.

In still another aspect, the invention contemplates a method for producing a differentiated transgenic plant, comprising introducing a chimeric DNA construct as broadly described above into regenerable plant cells so as to yield regenerable transformed cells, identifying or selecting a population of transformed cells, and regenerating a differentiated transgenic plant from said population.

In a preferred embodiment, the expression of the chimeric DNA construct renders the differentiated transgenic plant identifiable over the corresponding non-transgenic plant.

In a still further aspect, the invention provides a differentiated transgenic plant comprising plant cells containing a chimeric DNA construct as broadly described above.

The chimeric DNA construct is transmitted through a complete sexual cycle of said differentiated transgenic plant to its progeny so that it is expressed by the progeny plants. Thus, the invention also provides seed, other plant parts, tissue, and progeny plants derived from said differentiated transgenic plant.

### BRIEF DESCRIPTION OF THE DRAWINGS

FIG. 1. Structure of the ACT1 gene. Actin coding regions are denoted by shaded boxes and the 5′ untranslated leader (L) by open boxes. A horizontal arrow depicts the transcription start site while the intron within the 5′ leader is labelled Intron L. The sizes of coding exons (amino acid) are indicated above the diagram. The sizes of non-coding components (nt) are noted beneath the diagram.

FIG. 2. Characterisation of the ACT1 gene. (a) Primer extension analysis was carried out on total banana RNA using a 6-FAM labelled primer specific to the 5′UTR of ACT1. The primer sequence is given in the text and the size of primer extension products greater than 40 fluorescence units are shown under the trace. (b) Southern analysis of banana (Musa spp. cv. Bluggoe). Genomic DNA (10 μg) was digested with either EcoRI, PstI, BamHI or SacI and resolved on a 1.0% agarose gel, blotted to a nylon membrane, and hybridised with a 945 bp actin gene-specific probe from the 5′ flanking sequence. (c) Northern analysis of ACT1 transcript in major banana organs. Thirty micrograms of total RNA from leaves, roots, sepals/petals and stigma/stamen was resolved on a 1% agarose-formaldehyde gel, blotted onto nylon, and hybridised with a ACT1-specific probe from the 3′ untranslated region (upper panel). To observe relative loading of RNA, the membrane was stripped and reprobed with a 900 bp 18S rRNA specific probe (lower panel).

FIG. 3. Transient activity of ACT1 promoter truncations in banana embryogenic cells. (a) ACT1 promoter truncations fused to the uidA reporter gene were used in subsequent transient GUS assays. (b) GUS expression was assessed 48 h post-bombardment. The CaMV 35S promoter was included as a promoter comparison and untransformed (UT) banana tissue was included as a background control. Values are shown as mean GUS activity from 4 replicates±the standard error.

FIG. 4. Histochemical localisation of GUS expression in leaf and root sections of banana transformed with the Ba2.2-uidA gene fusion. GUS activity is indicated by an indigo dye precipitate after addition of X-gluc substrate and clearing in ethanol:acetic acid (1:5).

### BRIEF DESCRIPTION OF THE SEQUENCES: SUMMARY TABLE

TABLE A

| Sequence ID | | | |
|---|---|---|---|
| Number | Sequence | Length | |
| SEQ ID NO: 1 | Genomic sequence of actin gene obtained | 1610 | nts |
| | from Musa sp. | | |
| SEQ ID NO: 2 | Polypeptide product encoded by | 377 | aa |
| | SEQ ID NO: 1 | | |
| SEQ ID NO: 3 | Polynucleotide encoding promoter region | 2217 | nts |
| | designated Ba 2.2 | | |
| SEQ ID NO: 4 | Polynucleotide encoding promoter region | 1253 | nts |
| | designated Ba 1.2 | | |
| SEQ ID NO: 5 | Polynucleotide encoding promoter region | 1100 | nts |
| | designated Ba 1.1 | | |
| SEQ ID NO: 6 | Polynucleotide encoding promoter region | 902 | nts |
| | designated Ba 0.9 | | |
| SEQ ID NO: 7 | Polynucleotide encoding promoter region | 847 | nts |
| | designated Ba 0.8 | | |
| SEQ ID NO: 8 | LINKsac ™ primer | 26 | nts |
| SEQ ID NO: 9 | KNIL ™ primer | 22 | nts |
| SEQ ID NO: 10 | Actin-B primer | 20 | nts |
| SEQ ID NO: 11 | Actin-N primer | 18 | nts |
| SEQ ID NO: 12 | BACT1-B primer | 23 | nts |
| SEQ ID NO: 13 | BACT1-N primer | 18 | nts |
| SEQ ID NO: 14 | BACT15.0 primer | 21 | nts |
| SEQ ID NO: 15 | BACT13.0 primer | 21 | nts |
| SEQ ID NO: 16 | 6-FAM end-labelled primer | 27 | nts |
| SEQ ID NO: 17 | Ba5utrF primer | 22 | nts |
| SEQ ID NO: 18 | Actex3r primer | 20 | nts |
| SEQ ID NO: 19 | BaIF primer | 18 | nts |
| SEQ ID NO: 20 | Degenerate actin primer | 17 | nts |
| SEQ ID NO: 21 | Actex2f primer | 18 | nts |
| SEQ ID NO: 22 | Putative 3′ intron splice site | 10 | nts |
| SEQ ID NO: 23 | Monocot consensus 3′ intron splice site | 10 | nts |
| SEQ ID NO: 24 | Putative 5′ intron splice site | 8 | nts |
| SEQ ID NO: 25 | Monocot consensus 5′ intron splice site | 8 | nts |
| SEQ ID NO: 26 | G-box like motif | 6 | nts |
| SEQ ID NO: 27 | TATA-box like motif | 6 | nts |
| SEQ ID NO: 28 | Ba3utrF primer | 20 | nts |
| SEQ ID NO: 29 | Ba3utrR primer | 20 | nts |
| SEQ ID NO: 30 | 18Sf primer | 18 | nts |
| SEQ ID NO: 31 | 18Sr primer | 18 | nts |
| SEQ ID NO: 32 | Bact5.1 primer | 25 | nts |
| SEQ ID NO: 33 | Bact5.2 primer | 18 | nts |

nts = nucleotides;
aa = amino acids

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to sequences that vary by as much as 30%, preferably by as much as 20% and more preferably by as much as 10% to the length of a reference sequence.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically” and the like refers to antigen-binding molecules that bind the polypeptide or polypeptide fragments of the invention but do not significantly bind to homologous prior art polypeptides.

By “biologically active fragment” is meant a fragment that has at least about 0.1%, preferably at least about 10%, and more preferably at least about 25% of the activity of a reference promoter sequence. It will also be understood that the phrase “biologically active fragment” refers to a part of an indicated DNA sequence that initiates RNA transcription or that, when fused to a particular gene and introduced into a plant cell, causes expression of the gene at a level higher than is possible in the absence of such part of the indicated DNA sequence.

The terms “chimeric construct” or “chimeric DNA” and the like are used herein to refer to a gene or DNA sequence or segment comprising at least two DNA sequences or segments from species which do not combine DNA under natural conditions, or which DNA sequences or segments are positioned or linked in a manner which does not normally occur in the native genome of the untransformed plant.

Throughout this specification, unless the context requires otherwise, the words “comprise ”, “comprises” and “comprising” will be understood to imply the inclusion of a stated step or element or group of steps or elements but not the exclusion of any other step or element or group of steps or elements.

“Constitutive promoter” refers to a promoter that directs expression of an operably linked transcribable sequence in many or all tissues of a plant.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The terms “growing” or “regeneration” as used herein mean growing a whole, differentiated plant from a plant cell, a group of plant cells, a plant part (including seeds), or a plant piece (e.g., from a protoplast, callus, or tissue part).

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “marker gene” is meant a gene that imparts a distinct phenotype to cells expressing the marker gene and thus allows such transformed cells to be distinguished from cells that do not have the marker. A selectable marker gene confers a trait for which one can ‘select’ based on resistance to a selective agent (e.g., a herbicide, antibiotic, radiation, heat, or other treatment damaging to untransformed cells). A screenable marker gene (or reporter gene) confers a trait that one can identify through observatior or testing, i.e., by ‘screening’ (e.g.β-glucuronidase, luciferase, or other enzyme activity not present in untransformed cells).

By “obtained from ”is meant that a sample such as, for example, a nucleic acid extract is isolated from, or derived from, a particular source of the host. For example, the nucleic acid extract may be obtained from tissue isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant a functional linkage between, for example, a promoter sequence and an adjacent transcribable DNA sequence regulated by the promoter sequence. The operably linked promoter controls expression by modulating the transcription of the transcribable DNA sequence.

As used herein, “plant” and “differentiated plant” refer to a whole plant or plant part containing differentiated plant cell types, tissues and/or organ systems. Plantlets and seeds are also included within the meaning of the foregoing terms. Plants included in the invention are any plants amenable to transformation techniques, including angiosperms, gymnosperms, monocotyledons and dicotyledons.

The term “plant cell” as used herein refers to protoplasts, gamete-producing cells, and cells which regenerate into whole plants. Plant cells include cells in plants as well as protoplasts in culture.

By “plant tissue” is meant differentiated and undifferentiated tissue derived from roots, shoots, pollen, seeds, tumour tissue, such as crown galls, and various forms of aggregations of plant cells in culture, such as embryos and calluses.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” and the like refer to polynucleotides displaying substantial sequence identity with a reference polynucleotide sequence or polynucleotides that hybridise with a reference sequence under stringent conditions that are defined hereinafter. These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Accordingly, these terms encompass polynucleotides that initiate RNA transcription or that, when fused to a particular gene and introduced into a plant cell, cause expression of the gene at a level higher than is possible in the absence of such polynucleotides. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Typically, probes comprise at least 15 nucleotides, more suitably, at least 20 nucleotides, preferably at least 30 nucleotides, more preferably at least 50 nucleotides, even more preferably at least 100 nucleotides, even more preferably at least 200 nucleotides, even more preferably at least 400 nucleotides, even more preferably at least 600 nucleotides and still even more preferably at least 1000 nucleotides. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “promoter” is meant a sequence of nucleotides from which transcription of DNA operably linked downstream of said sequence (i.e., in the 3′ direction on the sense strand of double-stranded DNA) maybe initiated.

By “recombinant polypeptide” is meant a polypeptide made using recombinant techniques, i.e., through the expression of a recombinant polynucleotide.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation. The higher the stringency, the higher will be the degree of complementarity between immobilised nucleotide sequences and the labelled polynucleotide sequence.

“Stringent conditions” refers to temperature and ionic conditions under which only nucleotide sequences having a high frequency of complementary bases will hybridise. The stringency required is nucleotide sequence dependent and depends upon the various components present during hybridisation. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridises to a complementary probe.

The term “transcribable DNA sequence” or “transcribed DNA sequence”, excludes the non-transcribed regulatory sequence that drives transcription. Depending on the aspect of the invention, the transcribable sequence may be derived in whole or in part from any source known to the art, including a plant, a fungus, an animal, a bacterial genome or episome, eukaryotic, nuclear or plasmid DNA, cDNA, viral DNA or chemically synthesised DNA. A transcribable sequence may contain one or more modifications in either the coding or the untranslated regions, which could affect the biological activity or the chemical structure of the expression product, the rate of expression or the manner of expression control. Such modifications include, but are not limited to, insertions, deletions and substitutions of one or more nucleotides. The transcribable sequence may contain an uninterrupted coding sequence or it may include one or more introns, bound by the appropriate splice junctions. The transcribable sequence may also encode a fusion protein. It is contemplated that introduction into plant tissue of chimeric nucleic acid constructs of the invention will include constructions wherein the transcribable sequence and its promoter are each derived from different species.

The term “transformation ”means alteration of the genotype of a host plant by the introduction of a chimeric nucleic acid.

The term “transgene” is used herein to describe genetic material that has been or is about to be artificially inserted into the genome of a cell, particularly a plant cell. The transgene is used to transform a cell, meaning that a permanent or transient genetic change, preferably a permanent genetic change, is induced in a cell following incorporation of a chimeric DNA construct as defined herein. A permanent genetic change is generally achieved by introduction of the DNA into the genome of the cell.

As used herein, the term “transgenic” or “transformed” with respect to a plant cell, plant part (including seed), plant tissue or plant means a plant cell, plant part, plant tissue or plant which comprises an isolated chimeric DNA construct according to the invention which has been introduced into the nucleome, preferably the genome, of a plant cell, plant part, plant tissue or plant.

By “transgenote” is meant an immediate product of a transformation process.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, ie., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

The terms “wild type”, “native” or “non-transgenic” refers to an untransformed plant cell, plant part, plant tissue or plant, i.e., one where the nucleome, preferably the genome, has not been altered by the presence of a chimeric DNA construct as defined herein.

2. Transcribed DNA Sequence

The promoter of the present invention was discovered by its location adjacent to the start of an actin-encoding polynucleotide from banana (Musa spp.), whose sequence is set forth in SEQ ID NO: 1 and which is hereinafter referred to also as ACT1. SEQ ID NO: 1 is transcribed constitutively in vegetative tissues (e.g., leaves and stems) as well as reproductive tissues (e.g., petals/sepals and stigma/stamens) of banana. More particularly, SEQ ID NO: 1 is a genomic sequence extending 1610 nucleotides and comprising four exons and three introns. Exon 1 extends from position 1 through 71, exon 2 extends from position 161 through 552, exon 3 extends from position 628 through 1243, and exon 4 extends from 1348 through 1610. Intron 1 spans position 72 through 160, intron 2 spans position 553 through 627 and intron 3 spans position 1244 through 1347. A 5′ untranslated region (5′ UTR) is situated at position 1 to 11, and a 3′ untranslated region (3′ UTR) is located at position 1411 to 1610. A poly(A) tail is present at the end of this sequence. An open reading frame (ORF) is defined by positions 12 through 71, 161 through 552, 628 through 1243 and 1348 through 1410.

3. Polynucleotide Sequence Variants of Transcribed DNA Sequences

Suitable polynucleotide sequence variants of the invention may be prepared according to the following procedure:
(a) obtaining a nucleic acid extract from a suitable tissue of a plant, preferably a monocotyledonous plant and more preferably a non-graminaceous monocotyledonous plant;
(b) creating primers which are optionally degenerate wherein each comprises a portion of a reference polynucleotide corresponding to a transcribable DNA sequence of the invention; and
(c) using said primers to amplify, via nucleic acid amplification techniques, at least one amplification product from said nucleic acid extract, wherein said amplification product corresponds to a polynucleotide sequence variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996, J. Am. Chzem. Soc. 118:1587-1594 and International application WO 92/01813) and Lizardi et al., (International Application WO 97/19193) nucleic acid sequence-based amplification (NASBA) as for example described by Sooknanan et al., (1994, Biotechniques 17:1077-1080) which is incorporated herein by reference; and Q-β replicase amplification as for example described by Tyagi et al., (1996, Proc. Natl. Acad. Sci. USA 93:5395-5400).

Typically, polynucleotide sequence variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (“Molecular Cloning. A Laboratory Manual”, Cold Spring Harbour Press, 1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled addressee will recognise that a number of factors influence hybridisation. The specific activity of radioactively labelled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilized polynucleotide following washing. Washing ensures that the labelled polynucleotide is hybridised only to the immobilized polynucleotide with a desired degree of complementarity to the labelled polynucleotide.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 420° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.5 M to at least about 0.9 M salt for washing at 42° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 42° C.

Preferably, the polynucleotide variants hybridise to a reference polynucleotide under high stringency conditions. High stringency conditions include and encompass from at least about 31% v/v to at least about 50% v/v formamide and from at least about 0.01 M to at least about 0.15 M salt for hybridisation at 42° C., and at least about 0.01 M to at least about 0.15 M salt for washing at 42° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, washing is carried out at T=69.3+0.41 (G+C) %-12° C. However, the Tₘ of a duplex DNA decreases by 1° C. with every increase of 1% in the number of mismatched base pairs.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC/0.1% SDS for 15 min at 45° C., followed by 2×SSC/0.1% SDS for 15 min at 50° C.), followed by two sequential high stringency washes (i.e., 0.2×SSC/0.1% SDS for 12 min at 55° C. followed by 0.2×SSC and 0.1% SDS solution for 12 min).

Methods for detecting a labelled polynucleotide hybridised to an immobilised polynucleotide are well known to practitioners in the art. Such methods include autoradiography, phosphorimaging, and chemiluminescent, fluorescent and colorimetric detection.

4. Promoter Sequences of the Invention

4.1 Promoters Region of Transcribed DNA Sequence

The invention also provides a promoter isolated adjacent to the start of the transcribed DNA sequence described in Section 2. In particular, a constitutive promoter sequence for expression of chimeric or heterologous genes in plants, preferably monocotyledonous plants, is provided and is set forth in SEQ ID NO: 3. This promoter sequence is also referred to hereinafter as the ACT1 promoter.

The invention also contemplates biologically active portions of SEQ ID NO: 3 representative examples of which include the polynucleotide set forth in any one of SEQ ID NO: 4, 5, 6 and 7 as well as polynucleotide sequence variants thereof. Those of skill in the art will understand that a biologically active portion or fragment of a promoter sequence, when fused to a particular gene and introduced into a plant cell, causes expression of the gene at a level higher than is possible in the absence of such fragment. One or more biologically active fragments may be included in a promoter according to the present invention, for instance one or more motifs may be coupled to a “minimal” promoter. Such motifs may confer ACT1 promoter function on a promoter, such as suitability for enhanced performance in monocotyledonous plants and preferably non-graminaceous monocotyledonous plants such as, but not limited to, Musaceae (Milsa and Ensete), taro, ginger, onions, garlic, pineapple, bromeliaeds, palms, orchids, lilies, irises and the like.

The activity of a promoter can be determined by methods well known in the art. For example, the level of promoter activity is quantifiable by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as PCR. Use of a reporter gene facilitates determination of promoter activity by reference to protein production. Non-limiting methods for assessing promoter activity are disclosed by Medberry et al. (1992, Plant Cell 4:185; 1993, The Plant J. 3:619), Sambrook et al. (1989, supra) and McPherson et al. (U.S. Pat. No. 5,164,316).

4.2 Promoter Variants

Promoter variants that are substantially complementary to a reference promoter of the invention may be obtained by procedures outlined in Section 3.

In general, variants comprise regions that show at least 70%, more suitably at least 80%, preferably at least 90%, and most preferably at least 95% sequence identity over a reference promoter sequence of identical size (“comparison window”) or when compared to an aligned sequence in which the alignment is performed by a computer homology program known in the art. What constitutes suitable variants may be determined by conventional techniques. For example, polynucleotides according to SEQ ID NO: 3, 4, 5, 6 and 7 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a promoter of the invention. This technique is well known in the art as, for example, described by Adelman et al. (1983, DNA 2:183). Briefly, promoter DNA is altered by hybridising an oligonucleotide encoding the desired mutation to a template DNA, where the template is the single-stranded form of a plasmid or bacteriophage containing the unaltered or native DNA sequence of the promoter of interest. After hybridisation, a DNA polymerase is used to synthesise an entire second complementary strand of the template that will thus incorporate the oligonucleotide primer, and will code for the selected alteration in the promoter of interest.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridise properly to the single-stranded DNA template molecule.

The DNA template can be generated by those vectors that are either derived from bacteriophage M13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase I, is then added to synthesise the complementary strand of the template using the oligonucleotide as a primer for synthesis. A heteroduplex molecule is thus formed such that one strand of DNA encodes the mutated form of the promoter under test, and the other strand (the original template) encodes the native unaltered sequence of the promoter under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired promoter activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an Escherichia coli dut^{—}ung^{—}strain, which allows the incorporation of uracil into the template strand. The oligonucleotide is annealed to the template and extended with T4 DNA polymerase to create a double-stranded heteroduplex. Finally, the heteroduplex is introduced into a wild-type E. Coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct promoter variants according to the invention. In this regard, reference may be made, for example, to Ausubel et al., supra, in particular Chapters 8.2A and 8.5.

5. Chimeric DNA Construct

A promoter or biologically active variant or fragment according to the invention can be fused to a foreign or endogenous DNA sequence to create a chimeric DNA construct for introduction into plants.

5.1 3′ Non-Translated Region

In a preferred embodiment, the chimeric DNA construct of the present invention is in the form of an expression cassette designed for genetic transformation of plants. In this embodiment, the chimeric DNA construct suitably comprises a 3′ non-translated sequence that is operably linked to the foreign or endogenous DNA sequence and that functions in plant cells to terminate transcription and/or to cause addition of a polyadenylated nucleotide sequence to the 3′ end of a RNA sequence transcribed from the foreign or endogenous DNA sequence. Thus, a 3′ non-translated sequence refers to that portion of a gene comprising a DNA segment that contains a transcriptional termination signal and/ora polyadenylation signal and any other regulatory signals (e.g., translational termination signals) capable of effecting mRNA processing or gene expression. The polyadenylation signal is characterised by effecting the addition of polyadenylic acid tracts to the 3′ end of the mRNA precursor. Polyadenylation signals are commonly recognised by the presence of homology to the canonical form 5′ AATAAA-3′ although variations are not uncommon.

The 3′ non-translated regulatory DNA sequence preferably includes from about 50 to 1,000 nucleotide base pairs and contains plant transcriptional and translational termination sequences. Examples of suitable 3′ non-translated sequences are the 3′ transcribed non-translated regions containing a polyadenylation signal from the nopaline synthase (nos) gene of Agrobacterium tumefaciens Bevan et al., 1983, Nucl. Acid Res., 11:369) and the terminator for the T7 transcript from the octopine synthase gene of Agrobacterium tumefaciens. Alternatively, suitable 3′ non-translated sequences may be derived from plant genes such as the 3′ end of the protease inhibitor I or II genes from potato or tomato, the soybean storage protein genes and the pea E9 small subunit of the ribulose-1,5-bisphosphate carboxylase (ssRUBISCO) gene, although other 3′ elements known to those of skill in the art can also be employed. Alternatively, 3′ non-translated regulatory sequences can be obtained de novo as, for example, described by An (1987, Methods in Enzymology, 153:292).

5.2 Optional Sequences

The chimeric DNA construct of the present invention can further include enhancers, either translation or transcription enhancers, as may be required. These enhancer elements are well known to persons skilled in the art, and can include the ATG initiation codon and adjacent sequences. The initiation codon must be in phase with the reading frame of the coding sequence relating to the foreign or endogenous DNA sequence to ensure translation of the entire sequence. The translation control signals and initiation codons can be of a variety of origins, both natural and synthetic. Translational initiation regions may be provided from the source of the transcriptional initiation region, or from the foreign or endogenous DNA sequence. The sequence can also be derived from the source of the promoter selected to drive transcription, and can be specifically modified so as to increase translation of the mRNA.

Examples of transcriptional enhancers include, but are not restricted to, elements from the CaMV ³⁵S promoter and octopine synthase genes as for example described by Last et al. U.S. Pat. No. 5,290,924). It is proposed that the use of an enhancer element such as the ocs element, and particularly multiple copies of the element, will act to increase the level of transcription from adjacent promoters when applied in the context of plant transformation.

As the DNA sequence inserted between the transcription initiation site and the start of the coding sequence, i.e., the untranslated leader sequence, can influence gene expression, one can also employ a particular leader sequence. Preferred leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987, Nucl. Acid Res., 15:6643). However, other leader sequences, e.g., the leader sequence of RTBV, have a high degree of secondary structure that is expected to decrease mRNA stability and/or decrease translation of the mRNA. Thus, leader sequences (i) that do not have a high degree of secondary structure, (ii) that have a high degree of secondary structure where the secondary structure does not inhibit mRNA stability and/or decrease translation, or (iii) that are derived from genes that are highly expressed in plants, will be most preferred.

Regulatory elements such as the sucrose synthase intron as, for example, described by Vasil et al. (1989, Plant Physiol, 91:5175), the Adh intron I as, for example, described by Callis et al. (1987, Genes Develop., II), or the TMV omega element as, for example, described by Gallie et al. (1989, The Plant Cell, 1:301) can also be included where desired. Other such regulatory elements useful in the practice of the invention are known to those of skill in the art.

Additionally, targeting sequences may be employed to target a protein product of the foreign or endogenous DNA sequence to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. Thus, the chimeric DNA construct can further comprise a plastid transit peptide encoding DNA sequence operably linked between a promoter or biologically active variant or fragment according to the invention and the foreign or endogenous DNA sequence. For example, reference may be made to Heijne et al. (1989, Eur. J. Biochem., 180:535) and Keegstra et al. (1989, Ann. Rev. Plant Physiol. Plant Mol. Biol., 40:471).

A chimeric DNA construct can also be introduced into a vector, such as a plasmid. Plasmid vectors include additional DNA sequences that provide for easy selection, amplification, and transformation of the expression cassette in prokaryotic and eukaryotic cells, e.g., pUC-derived vectors, pSK-derived vectors, pGEM-derived vectors, pSP-derived vectors, or pBS-derived vectors. Additional DNA sequences include origins of replication to provide for autonomous replication of the vector, selectable marker genes, preferably encoding antibiotic or herbicide resistance, unique multiple cloning sites providing for multiple sites to insert DNA sequences or genes encoded in the chimeric DNA construct, and sequences that enhance transformation of prokaryotic and eukaryotic cells.

The vector preferably contains an element(s) that permits stable integration of the vector into the host cell genome or autonomous replication of the vector in the cell independent of the genome of the cell. The vector may be integrated into the host cell genome when introduced into a host cell. For integration, the vector may rely on the foreign or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAM.beta.1 permitting replication in Bacillus. The origin of replication may be one having a mutation to make its function temperature-sensitive in a Bacillus cell (see, e.g., Ehrlich, 1978, Proc. Natl. Acad. Sci. USA 75:1433).

5.3 Marker Genes

To facilitate identification of transformants, the chimeric DNA construct desirably comprises a selectable or screenable marker gene as, or in addition to, the expressible foreign or endogenous DNA sequence. The actual choice of a marker is not crucial as long as it is functional (i.e., selective) in combination with the plant cells of choice. The marker gene and the foreign or endogenous DNA sequence of interest do not have to be linked, since co-transformation of unlinked genes as, for example, described in U.S. Pat. No. 4,399,216 is also an efficient process in plant transformation.

Included within the terms selectable or screenable marker genes are genes that encode a “secretable marker” whose secretion can be detected as a means of identifying or selecting for transformed cells. Examples include markers that encode a secretable antigen that can be identified by antibody interaction, or secretable enzymes that can be detected by their catalytic activity. Secretable proteins include, but are not restricted to, proteins that are inserted or trapped in the cell wall (e.g., proteins that include a leader sequence such as that found in the expression unit of extensin or tobacco PR-S); small, diffusible proteins detectable, e.g. by ELISA; and small active enzymes detectable in extracellular solution (e.g., α-amylase, β-lactamase, phosphinothricin acetyltransferase).

5.4 Selectable Markers

Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (neo) gene conferring resistance to kanamycin, paromomycin, G418 and the like as, for example, described by Potrykus et al. (1985, Mol. Gen. Genet. 199:183); a glutathione-S-transferase gene from rat liver conferring resistance to glutathione derived herbicides as, for example, described in EP-A 256 223; a glutamine synthetase gene conferring, upon overexpression, resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A 275 957, a gene encoding a 5-enolshikimate-3-phosphate synthase (EPSPS) conferring tolerance to N-phosphonomethylglycine as, for example, described by Hinchee et al. (1988, Biotech., 6:915), a bar gene conferring resistance against bialaphos as, for example, described in WO91/02071; a nitrilase gene such as bxn from Klebsiella ozaenae which confers resistance to bromoxynil (Stalker et al., 1988, Science, 242:419); a dihydrofolate reductase (DHFR) gene conferring resistance to methotrexate (Thillet et al., 1988, J. Biol. Chem., 263:12500); a mutant acetolactate synthase gene (ALS), which confers resistance to imidazolinone, sulfonylurea or other ALS-inhibiting chemicals (EP-A-154 204); a mutated anthranilate synthase gene that confers resistance to 5-methyl tryptophan; or a dalapon dehalogenase gene that confers resistance to the herbicide.

5.5 Screenable Markers

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985, Biochem. Biophys. Res. Comm., 126:1259), which may be employed in calcium-sensitive bioluminescence detection; a green fluorescent protein gene (Niedz et al., 1995 Plant Cell Reports, 14:403); a luciferase (luc) gene (Ow et al., 1986, Science, 234:856), which allows for bioluminescence detection; a α-lactamase gene (Sutcliffe, 1978, Proc. Natl. Acad. Sci. USA 75:3737), which encodes an enzyme for which various chromogenic substrates are known (e.g., PADAC, a chromogenic cephalosporin); an R-locus gene, encoding a product that regulates the production of anthocyanin pigments (red color) in plant tissues (Dellaporta et al., 1988, in Chromosome Structure and Function, pp. 263-282); an α-amylase gene (Ikuta et al., 1990, Biotech., 8:241); a tyrosinase gene (Katz et al., 1983, J. Gen. Microbiol., 129:2703) which encodes an enzyme capable of oxidizing tyrosine to dopa and dopaquinone which in turn condenses to form the easily detectable compound melanin; or a xylE gene (Zukowsky et al., 1983, Proc. Natl. Acad. Sci. USA 80:1101), which encodes a catechol dioxygenase that can convert chromogenic catechols.

6. Uses of the Promoter of the Invention

The isolated promoter sequence may be used, inter alia, to drive expression of a foreign or endogenous DNA sequence. The foreign or endogenous DNA sequence may comprise a region transcribed into an RNA molecule that modulates the expression of a corresponding target gene. Such modulation of expression may be effected, for example, by antisense technology, ribozyme technology and co-suppression or homology-dependent gene silencing as is known in the art. Thus, the transcript may comprise an antisense RNA or a ribozyme or other transcript aimed at downregulation of expression of the corresponding target gene.

Alternatively, the foreign or endogenous DNA sequence may encode a detectable or measurable product, e.g. β-glucuronidase or luciferase; a selectable product, e.g., neomycin phosphotransferase (nptII) conferring resistance to aminoglycosidic antibiotics such as geneticin and paromomycin; a product conferring herbicide tolerance, e.g. glyphosate resistance or glufosinate resistance; a product affecting starch biosynthesis or modification e.g. starch branching enzyme, starch synthases, ADP-glucose pyrophosphorylase; a product involved in fatty acid biosynthesis, e.g. desaturase or hydroxylase; a product conferring insect resistance, e.g. crystal toxin protein of Bacillus thuringientsis; a product conferring viral resistance, e.g. viral coat protein; a product conferring fungal resistance, e.g. chitinase, β-1,3-glucanase or phytoalexins; a product altering sucrose metabolism, e.g. invertase or sucrose synthase; a gene encoding valuable pharmaceuticals, e.g. antibiotics, secondary metabolites, pharmaceutical peptides or vaccines.

The foreign or endogenous DNA sequence includes, but is not limited to, DNA from plant genes, and non-plant genes such as those from bacteria, yeasts, animals or viruses. Moreover, it is within the scope of the invention to isolate a foreign or endogenous DNA sequence from a given plant genotype, and to subsequently introduce multiple copies of that sequence into the same genotype, e.g., to enhance production of a given gene product. The introduced DNA can include modified genes, portions of genes, or chimeric genes, including genes from the same or different plant genotype.

Preferred agronomic properties encoded by the foreign or endogenous DNA sequence include, but are not limited to: traits that are beneficial to the grower such as resistance to water deficit, pest resistance or tolerance, herbicide resistance or tolerance, disease resistance or tolerance (e.g., resistance to viruses or fungal pathogens), stress tolerance (increased salt tolerance) and improved food content or increased yields; traits that are beneficial to the consumer of the horticultural produce harvested from the plant such as improved nutritive content in human food or animal feed; or beneficial to the food processor such as improved processing traits. In such uses, the transgenic plants containing the promoter of the invention are generally grown for the use of their grain, fruit and other plant parts, including stalks, husks, vegetative parts, and the like in human or animal foods including use as part of animal silage or for ornamental purposes. Often, chemical constituents of crops are extracted for foods or industrial use and transgenic plants may be created which have enhanced or modified levels of such components.

The isolated promoter sequence of the invention may also find use in the commercial manufacture of proteins or other compounds, where the compound of interest is extracted or purified from plant parts, seeds, and the like. Such proteins or compounds include, but are not limited to, immunogenic molecules for use in vaccines, cytokines and hormones. Cells or tissue from the plants may also be cultured, grown in vitro, or fermented to manufacture such molecules.

The transgenic plants containing the isolated promoter sequence of the invention may also be used in commercial breeding programs, or may be crossed or bred to plants of related crop species. Improvements encoded by the foreign or endogenous DNA sequence may be transferred, e.g., from cells of one plant species to cells of another plant species, e.g., by protoplast fusion.

The transgenic plants containing the isolated promoter sequence of the invention may have many uses in research or breeding, including creation of new mutant plants through insertional mutagenesis, in order to identify beneficial mutants that might later be created by traditional mutation and selection. An example would be the introduction of a recombinant DNA sequence encoding a transposable element that may be used for generating genetic variation or the introduction of unique “signature sequences” or other marker sequences which can be used to identify proprietary lines or varieties.

7. Introduction of Chimeric Construct into Plant Cells

Generally, the present invention employs recipient plant cells that are susceptible to transformation and subsequent regeneration into stably transformed, fertile plants. For monocot transformation for example, immature embryos, meristematic tissue, gametic tissue, embryogenic suspension cultures or embryogenic callus tissue can be employed as a source of recipient cells which is useful in the practice of the invention. For dicot transformation, organ and tissue cultures can be employed as a source of recipient cells. Thus, tissues, e.g., leaves, seed and roots, of dicots can provide a source of recipient cells useful in the practice of the invention.

Cultured susceptible recipient cells are preferably grown on solid supports. Nutrients are provided to the cultures in the form of media and the environmental conditions for the cultures are controlled. Media and environmental conditions which support the growth of regenerable plant cultures are well known to the art.

A number of techniques are available for the introduction of DNA into a recipient plant cell. There are many plant transformation techniques well known to workers in the art, and new techniques are continually becoming known. The particular choice of a transformation technology will be determined by its efficiency to transform certain plant species as well as the experience and preference of the person practising the invention with a particular methodology of choice. It will be apparent to the skilled person that the particular choice of a transformation system to introduce a chimeric DNA construct into plant cells is not essential to or a limitation of the invention, provided it achieves an acceptable level of nucleic acid transfer. Guidance in the practical implementation of transformation systems for plant improvement is provided, for example, by Birch (1997, Annu. Rev. Plant Physiol. Plant Molec. Biol. 48: 297-326).

In principle both dicotyledonous and monocotyledonous plants that are amenable to transformation, can be modified by introducing a chimeric DNA construct according to the invention into a recipient cell and growing a new plant that harbours and expresses the foreign or endogenous DNA sequence.

Introduction and expression of foreign or chimeric DNA sequences in dicotyledonous (broad-leafed) plants such as tobacco, potato and alfalfa has been shown to be possible using the T-DNA of the tumour-inducing (Ti) plasmid of Agrobacterium tumefaciens (See, for example, Umbeck, U.S. Pat. No. 5,004,863, and International application PCT/US93/02480). A construct of the invention may be introduced into a plant cell utilising A. tumefaciens containing the Ti plasmid. In using an A. tumefaciens culture as a transformation vehicle, it is most advantageous to use a non-oncogenic strain of the Agrobacterium as the vector carrier so that normal non-oncogenic differentiation of the transformed tissues is possible. It is preferred that the Agrobacterium harbours a binary Ti plasmid system. Such a binary system comprises (1) a first Ti plasmid having a virulence region essential for the introduction of transfer DNA (T-DNA) into plants, and (2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells as, for example, described by De Framond (1983, Biotechnology, 1:262) and Hoekema et al. (1983, Nature, 303:179). Such a binary system is preferred inter alia because it does not require integration into the Ti plasmid in Agrobacterium.

Methods involving the use of Agrobacterium include, but are not limited to: (a) co-cultivation of Agrobacterium with cultured isolated protoplasts; (b) transformation of plant cells or tissues with Agrobacterium; or (c) transformation of seeds, apices or meristems with Agrobacterium.

Recently, rice and corn, which are monocots, have been shown to be susceptible to transformation by Agrobacterium as well. Garlic and onion, which are also monocots, have been successfully transformed and regenerated by Agrobacterium mediated gene transfer (Kondo et al., 2000, Plant Cell Reports, 19(10): 989-993; Eady et al., 2000, Plant Cell Reports, 19(4): 376-381). However, many other important monocot crop plants, including oats, sorghum, millet, and rye, have not yet been successfully transformed using Agrobacterium-mediated transformation. The Ti plasmid, however, may be manipulated in the future to act as a vector for these other monocot plants. Additionally, using the Ti plasmid as a model system, it may be possible to artificially construct transformation vectors for these plants. Ti plasmids might also be introduced into monocot plants by artificial methods such as microinjection, or fusion between monocot protoplasts and bacterial spheroplasts containing the T-region, which can then be integrated into the plant nuclear DNA.

In addition, gene transfer can be accomplished by in situ transformation by Agrobacterium, as described by Bechtold et al. (1993, CR. Acad. Sci. Paris, 316:1194). This approach is based on the vacuum infiltration of a suspension of Agrobacterium cells.

Alternatively, foreign or chimeric nucleic acids may be introduced using root-inducing (Ri) plasmids of Agrobacterium as vectors.

Cauliflower mosaic virus (CaMV) may also be used as a vector for introducing of exogenous nucleic acids into plant cells (U.S. Pat. No. 4,407,956). CaMV DNA genome is inserted into a parent bacterial plasmid creating a recombinant DNA molecule that can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

Foreign or chimeric nucleic acids can also be introduced into plant cells by electroporation as, for example, described by Fromm et al. (1985, Proc. Natl. Acad. Sci., U.S.A, 82:5824) and Shimamoto et al. (1989, Nature 338:274-276). In this technique, plant protoplasts are electroporated in the presence of vectors or nucleic acids containing the relevant nucleic acid sequences. Electrical impulses of high field strength reversibly permeabilise membranes allowing the introduction of nucleic acids. Electroporated plant protoplasts reform the cell wall, divide and form a plant callus.

Another method for introducing foreign or chimeric nucleic acids into a plant cell is high velocity ballistic penetration by small particles (also known as particle bombardment or microprojectile bombardment) with the nucleic acid to be introduced contained either within the matrix of small beads or particles, or on the surface thereof as, for example described by Klein et al. (1987, Nature 327:70). Although typically only a single introduction of a new nucleic acid sequence is required, this method particularly provides for multiple introductions.

Alternatively, foreign or chimeric nucleic acids can be introduced into a plant cell by contacting the plant cell using mechanical or chemical means. For example, a nucleic acid can be mechanically transferred by microinjection directly into plant cells by use of micropipettes. Alternatively, a nucleic acid may be transferred into the plant cell by using polyethylene glycol which forms a precipitation complex with genetic material that is taken up by the cell.

There are a variety of methods known currently for transformation of monocotyledonous plants. Presently, preferred methods for transformation of monocots are microprojectile bombardment of explants or suspension cells, and direct DNA uptake or electroporation as, for example, described by Shimamoto et al. (1989, supra). Transgenic maize plants have been obtained by introducing the Streptomyces hygroscopicus bar gene into embryogenic cells of a maize suspension culture by microprojectile bombardment (Gordon-Kamm, 1990, Plant Cell, 2:603-618). The introduction of genetic material into aleurone protoplasts of other monocotyledonous crops such as wheat and barley has been reported (Lee, 1989, Plant Mol. Biol. 13:21-30). Wheat plants have been regenerated from embryogenic suspension culture by selecting only the aged compact and nodular embryogenic callus tissues for the establishment of the embryogenic suspension cultures (Vasil, 1990, Bio/Technol. 8:429-434). The combination with transformation systems for these crops enables the application of the present invention to monocots. These methods may also be applied for the transformation and regeneration of dicots. Transgenic sugarcane plants have been regenerated from embryogenic callus as, for example, described by Bower et al. (1996, Molecular Breeding 2:239-249).

Alternatively, a combination of different techniques may be employed to enhance the efficiency of the transformation process, e.g., bombardment with Agrobacterium coated microparticles (EP-A-486234) or microprojectile bombardment to induce wounding followed by co-cultivation with Agrobacterium (EP-A-486233).

8. Production and Characterisation of Differentiated Transgenic Plants

8.1 Regeneration

The methods used to regenerate transformed cells into differentiated plants are not critical to this invention, and any method suitable for a target plant can be employed. Normally, a plant cell is regenerated to obtain a whole plant following a transformation process.

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilised include auxins and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Transformation can be performed in the context of organ or plant part regeneration as, for example, described in Methods in Enzymology, Vol. 118 and Klee et al. (1987, Annual Review of Plant Physiology, 38:467), which are incorporated herein by reference. Utilising the leaf disk-transformation-regeneration method of Horsch et al. (1985, Science, 227:1229), disks are cultured on selective media, followed by shoot formation in about 24 weeks. Shoots that develop are excised from calli and transplanted to appropriate root-inducing selective medium. Rooted plantlets are transplanted to soil as soon as possible after roots appear. The plantlets can be repotted as required, until maturity is reached.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use.

In seed propagated crops, the mature transgenic plants can be self-crossed to produce a homozygous inbred plant. The inbred plant produces seed containing the newly introduced foreign gene(s). These seeds can be grown to produce plants that would produce the selected phenotype, e.g., early flowering.

The transgenic plants of the invention include, but are not limited to, a transgenic T0 or R0 plant, i.e., the first plant regenerated from transformed plant cells, a transgenic T1 or R1 plant, i.e., the first generation progeny plant, and progeny plants of further generations derived therefrom which comprise and express the chimeric DNA construct.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

It will be appreciated that the literature describes numerous techniques for regenerating specific plant types and more are continually becoming known. Those of ordinary skill in the art can refer to the literature for details and select suitable techniques without undue experimentation.

9. Characterisation

To confirm the presence of the foreign or endogenous DNA sequence in the regenerating plants, a variety of assays may be performed. Such assays include, for example, “molecular biological” assays well known to those of skill in the art, such as Southern and Northern blotting and PCR. A protein expressed by the heterologous DNA may be analysed by high performance liquid chromatography or ELISA (e.g., nptII) as is well known in the art.

In order that the invention may be readily understood and put into practical effect, particular preferred embodiments will now be described by way of the following non-limiting examples.

### EXAMPLES

### Example 1

Isolation and Cloning of the Act1 Promoter

The ACT1 promoter was isolated using a combination of ligation-mediated PCR (Mueller and Wold, 1989) and a method for amplifying flanking sequences as described below. Banana (Musa spp. cv. Bluggoe) genomic DNA was isolated from leaves of 2-3 month old in vitro plantlets essentially as described by Stewart and Via (1993). Genomic DNA (100 ng) was digested with SacI 37° C. for 12 h and the restriction enzyme inactivated by incubation at 68° C./10 min. A linker (50 pmol), created by annealing the LINKsac™ primer (5′-AGAATTCTGCAGGATCCCGGGGAGCT-3′ [SEQ ID NO: 8]) and KNIL™ primer (5′-CCCCGGGATCCTGCAGAATTCG-3′ [SEQ ID NO: 9]; 5′ phosphorylated and 3′ amino blocked), was ligated to the digested DNA at 14° C./12 h. The ligation product was used as template for a PCR with 200 μM of each dNTP, 5 μL Buffer 3 (Expand™ Long Template PCR System; Roche), 20 pmol of primers actin-N (5′-ACCTTGACCATTCCAGTGCC-3′ [SEQ ID NO: 10]) and LINKsac™, 0.5 U Expand polymerase (Expand™ Long Template PCR System; Roche) and ddH₂O to final volume of 50 μL. The reaction mix was subjected to PCR cycles of, 1 cycle: 92° C., 2 min, 35 cycles: 92° C., 30 s: 50° C., 30 s: 68° C., 3 min, 1 cycle: 68° C., 10 min and 4° C. soak. A final nested PCR was carried out on the resulting products using primers, actin-N (5′-AGTGCCATTGTCACAGAC-3′ [SEQ ID NO: 11]) and LINKsac™, under similar conditions.

A second amplification reaction was carried out using a method for amplifying flanking sequences, with primers designed to the 5′ sequence of the product isolated by ligation-mediated PCR. In brief, 50 ng-1 μg of genomic DNA was mixed with 20 pmol of a 5′ biotinylated primer, BACT1-B (5′-GATACGTGTTGCGGATCCCACAG-3′ [SEQ ID NO: 12]), and cycled as previously described. The products from this primer extension were purified using Dynabeads® (Dynabeads® Kilobase BINDER™ kit) according to manufacturer's directions. In summary, 5 μL of beads were washed twice in Binding Buffer (provided by the manufacturer) and resuspended in 20 μL of Binding Buffer. To this, 20 μL of the extension product was added and incubated for 3 h at 25° C. with gentle agitation. Template genomic DNA was removed by washing in 0.2 M NaOH (lx 30 min, and 4×1 min), TE Buffer (5×1 min) and ddH₂O (5×1 min) and the purified beads resuspended in 20 μL of ddH₂O. A PCR was carried out with the BACT1-N primer (5′-ACGGAAGTCGAATATGCC-3′ [SEQ ID NO: 13]) using conditions similar to the BACT1-B extension, except 1 μL of purified template was added instead of genomic DNA. The putative upstream actin sequence was amplified using an overlapping PCR approach with primers BACT15.0 (5′-ACCTTAGTCTGAGAGCTCTGA-3′ [SEQ ID NO: 14]) and BACT13.0 (5′-GTTATGGATATCTGCAAAACC-3′ [SEQ ID NO: 15]) using similar conditions to BACT1-B extension. The subsequent PCR product was cloned into pGEM-T (Promega) and sequenced using the ABI PRISM® BigDye™ Terminator system (PE Biosystems).

### Example 2

Mapping of the Traniscriptional Start Site, Leader Intron, Polyadenylation Site and Actin Gene Isolation

The precise 5′ end of ACT1 mRNA was mapped using primer extension. Total RNA from banana embryogenic cells was isolated essentially as described by Chang et al., (1993). Total RNA (20 μg) was annealed to 10 pmol of 6-FAM end-labelled primer (synthesised by Genset, Lismore, Australia) 5′-GTCAGCCATGTTATGGATATCTT ACAC-3′ [SEQ ID NO: 16], for 10 min at 75° C. The reagents for cDNA synthesis were added on ice (10 mM DTT, 1 mM each dNTP, 20 U RNase inhibitor, 40 U Expand reverse transcriptase in buffer (Roche)) and the reaction incubated for 90 min at 42° C. Following removal of RNA by incubation with 25 ng DNase-free RNase (Roche) for 30 min at 37° C., cDNA was precipitated in 0.3 M sodium acetate with 2.5 volumes of ethanol. The primer extension products were resuspended in 8 μL 95% [v/v] formamide, 10 mM EDTA (pH9.0) and electrophoresed in a 6 M urea, 4.5% polyacrylamide TBE (45 mM Tris-borate, 1 mM EDTA, pH 8.0) gel. Fluorescent signal was detected using an ABI 337 with GeneScan™ analysis software, and the size of the cDNA extension product determined by comparison with GS 500Rox molecular size markers (PE Biosystems).

The intron within the 5′ untranslated leader (UTL) sequence was mapped using a PCR strategy described by An et al., (1996a). A sense primer (Ba5utrF; 5′-CACCTCTCACTCTCCATCTCTC-3′ [SEQ ID NO: 17]) and anti-sense primer (Actex3r; 5′-CACTTCATGATGGAGTTGTA-3′ [SEQ ID NO: 18]) were used to PCR amplify the RNA leader-intron junction from Actex3r-primed cDNA. RT-PCR was carried out essentially as described for primer extension, except 1 μg of total RNA was used. The leader intron and splice sites were mapped by comparing the cDNA sequence of the PCR product with the previously obtained genomic sequence.

The ACT1 gene was amplified from genomic DNA using an ACT, promoter specific primer BaIF (5′-GTTCTTCCTCCTTCGATT-3′ [SEQ ID NO: 19]) and a degenerate actin primer (5′-TAGAAGCACTTCATGTG-3′ [SEQ ID NO: 20]) complementary to the 3′ end of exon 4. The 3′ untranslated region (UTR) of ACT1 was isolated using a 3′ RACE approach (Frohman et al., 1988). PCR was carried out with primers Actex2f (5′-CCCTGAGGAGCACCCTGT-3′ [SEQ ID NO: 21]) and oligo-dT(5′-NT₂₀) using oligo-dT primed cDNA as template. PCR products were cloned into pGEM-T (Promega) and sequence verified as described earlier.

### Example 3

Sequence Analysis

Using the strategy outlined in Example 1, a 1.2 kb fragment was amplified from banana genomic DNA. Sequence analysis of this product revealed strong homology to the rice actin exon 1 sequence (McElroy et al., 1990c). To obtain further upstream sequences primers were designed to the 5′ end of this fragment and the flanking sequences amplified. This resulted in a single 1 kb product and subsequent Southern hybridisation with an ACT1-specific oligonucleotide probe indicated the product was specific. Cloning and sequencing confirmed this and the ACT1 promoter sequence (2.2 kb) was assembled from these two fragments using overlapping PCR.

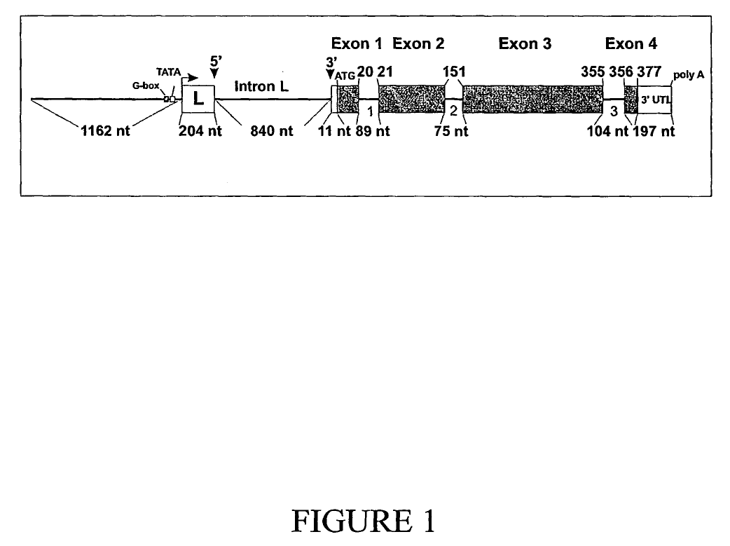

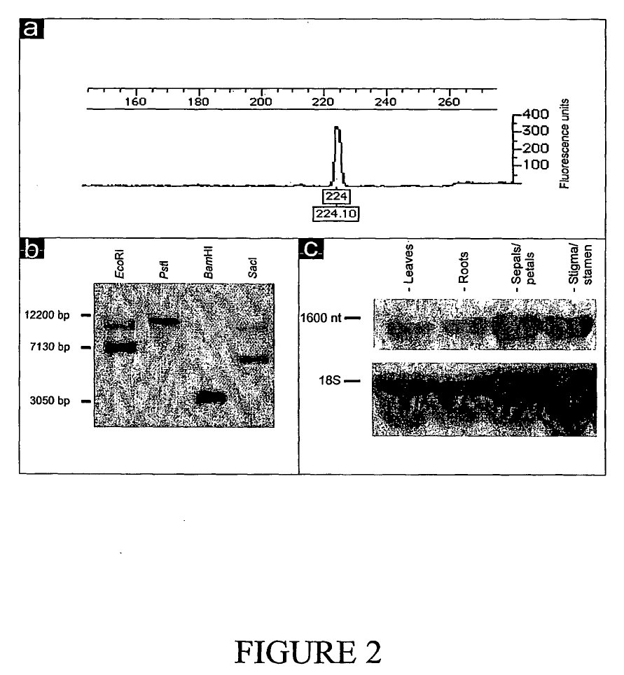

Alignment of the 3′ end of the ACT1 promoter with other actin promoters revealed the presence of a putative 3′ intron splice site (TTTTGCAG/AT [SEQ ID NO: 22]) which was almost identical to the monocot consensus (TTTTGCAG/GT [SEQ ID NO: 23]) (Simpson and Filipowicz, 1996). The precise location of the intron was mapped using a RT-PCR approach targeted to the 5′ UTL (see Example 2). Comparison of the cDNA and genomic sequences revealed that the ACT1 5′ UTL contained a relatively large intron (840 bp) which was spliced 11 nt upstream from the translation start codon (FIG. 1). Analysis of the 5′ intron splice site (AG/GTCAGT [SEQ ID NO: 24]), showed that, like the 3′ splice site, it was similar to the monocot consensus (AG/GTAAGT [SEQ ID NO: 25]) (Simpson and Filipowicz, 1996). The ACT1 transcriptional start site was mapped by primer extension using a fluorescently labelled oligonucleotide. A single product of 224 bp was generated (FIG. 2a) and mapped to position −1055 from the translational start site, indicating the ACT1 gene has a 215 nt 5′ untranslated leader (UTL) sequence. Like other plant actin 5′ UTLs, this sequence contained a number of C+T rich stretches and had a high overall pyrimidine content (approximately 65%). Analysis of the sequence upstream from the transcriptional start site using published regulatory element databases (Rombauts et al., 1999; Wingender et al., 1996) identified a G-box like motif (CACGTA [SEQ ID NO: 26]) and a TATA-box like motif (TTAATA [SEQ ID NO: 27]) located 98 nt and 60 nt upstream from the transcription start site, respectively.

The ACT1 gene was amplified from both genomic DNA and cDNA using ACT1-specific primers designed to the promoter region in combination with degenerate actin primers and 3′ RACE to amplify the 3′ UTR. The ACT1 gene potentially encoded a protein of 377 amino acids and contained three small introns at identical locations to most other plant actins. Intron 1 (89 bp) separated codons 20 and 21, intron 2 (75 bp) split codon 151 and intron 3 (104 bp) separated codons 356 and 357. One ACT1 polyadenylation site was mapped 197 bp downstream of the stop codon, however, the existence of additional poly(A) sites was not determined. Homology searches on the potential gene product using BLASTP (Altschul et al., 1997) revealed that the amino acid sequence was highly homologous to the vegetatively expressed Arabidopsis thaliana ACT7 protein (8 amino acid changes, 4 non-conservative). Furthermore, the putative ACT1 gene product contained residues common to vegetatively expressed actins (An et al., 1999) including valine-219, serine-232 and serine-358 and lacked serine-79 which is conserved in Arabidopsis actins expressed in mature pollen (Kandasamy et al., 1999).

### Example 4

Genomic and Expression Analysis of the ACT1 Gene

Genomic DNA (10 μg) was isolated from banana embryogenic cells as previously described and digested independently with BamHI, PstI, EcoRI or SacI. Digests were electrophoresed in a 1.0% agarose gel, capillary blotted onto a nylon membrane (Roche) and baked for 2 h at 80° C. Prior to hybridisation, the membrane was blocked for 60 min at 42° C. with DIG Easy Hyb (Roche). DIG-labelled probes were PCR amplified using a mixture of DIG-labelled and standard dNTPS (1:9 ratio). Two ACT1 probes were generated, (i) a 945 bp 5′ flanking sequence probe complementary to nt −945 to +1, using primers Ba5utrF and BACT13.0 and (ii) a 210 bp 3′ UTR probe complementary to nt +1387 to +1596 using primers Ba3utrF (5′-CAGGAAGTGCTTCTGAGTTC-3′ [SEQ ID NO: 28]) and Ba3utrR (5′-ATAAACAGCCTTCATTGCAG-3′ [SEQ ID NO: 29]). The membrane was hybridised with DIG-labelled probes for 12 h at 42° C. followed by two washes at room temperature (10 min) in 2×SSC/0.1% SDS and two washes at 65° C. (15 min) in 0.1×SSC/0.1% SDS. Detection of hybridised probe using CDP-STAR™(Roche) was carried out according to the manufacturer's instructions. Localisation of ACT1 gene expression was assessed by northern analysis. Total RNA was extracted from leaves, roots and flowers of mature banana plants as previously described. RNA samples (30 μg) were electrophoresed in an agarose-formaldehyde gel (Sambrook et al. 1989) and capillary blotted onto a nylon membrane (Roche). The membranes were hybridised in a manner similar to the Southern hybridisation, using the same 3′ UTR ACT1 DIG-labelled probe. To ensure even loading of RNA, the membrane was stripped using two 15 min washes in 0.1% SDS and reprobed with a 900 bp 18S ribosomal DIG-labelled probe, PCR amplified with primers 18Sf (5′-CAGACTGTGAAACTGCGA-3′ [SEQ ID NO: 30]) and 18Sr (5′-GCTTTCGCAGTGGTTCGT-3′ [SEQ ID NO: 31]) as previously described.

From the foregoing, banana genomic DNA was extracted and used in Southern hybridisation with an ACT1 promoter-specific probe to ascertain the presence of genes closely related to ACT1. Either one or two bands hybridised with the probe depending on the restriction enzyme used (FIG. 2b). Similar results were also obtained using an ACT1-3′ UTR specific probe (results not shown). As none of the restriction enzyme sites were present within the probe sequence, the hybridisation pattern observed is probably due to allelic differences in either the ACT1 gene and/or adjacent sequences. Northern hybridisation was subsequently used to investigate the distribution and level of ACT1 mRNA in various banana tissues. The ACT1 3′ UTR-specific probe was used to detect ACT1 transcript in total RNA isolated from leaves, roots and petals/sepals, stigma/stamens from flowers (FIG. 2c). To observe the relative amounts of RNA loaded, the blot was stripped and reprobed with an 18S ribosomal specific probe. Northern analysis revealed the presence of a 1600 nt ACT1-specific transcript which was similar in size to those from Arabidopsis (An et al. 1996a; An et al. 1996b; McDowell et al. 1996b). The ACT1-specific transcript was present in all tissues tested and, although equal amounts of RNA were not loaded, it appeared that ACT1 transcript levels were approximately equal between tissues.

### Example 5

Construction of uidA Reporter Fusions

A 2217 bp putative promoter region (Ba2.2) was excised from the pGEM-ACT1 clone as a SacII/NcoI fragment and inserted upstream of the uidA reporter gene (encoding β-glucuronidase) and the tobacco ribulose-1,5-bisphosphate carboxylase/oxygenase small subunit gene 3′ UTR in pUC19. This construct was designated pBa2.2-uidA. Three promoter truncations, Ba1.2 (1253 bp), Ba1.1(1100 bp) and Ba0.9 (902 bp), were PCR amplified as previously described using primers Bact5.1 (5′-CGAGACAGCTCATTGACGAACACAA-3′ [SEQ ID NO: 32), Bact5.2 (5′-TTTAAGCTGTCTCGTCGC-3′ [SEQ ID NO: 33) or Ba5utrF, respectively, and BACT13.0. The PCR products were cloned upstream of uidA as described for Ba2.2-uidA, and designated pBa1.2-uidA, pBa1.1-uidA and pBa0.9-uidA, respectively. A further truncation which removed the 5′ intron splice site was made by digesting Ba2.2 with Sal/I/NcoI. The resulting 847 bp product (Ba0.8) was inserted upstream of the uidA reporter gene in pBa0.9-uidA, creating pBa0.8-uidA. An 800 bp cauliflower mosaic virus (CaMV) 35S promoter aid the maize polyubiquitin promoter (ubil), driving uidA were used to compare promoter expression (Dugdale et al. 1998; Christensen and Quail, 1996). Plasmid DNA for microprojectile bombardment was prepared using a Bresapure™ Plasmid Maxi Kit according to the manufacturer's instructions (Geneworks).

### Example 6

Analysis of Promoter Strength

GUS activity was measured both histochemically and fluorometrically essentially as described by Jefferson et al., (1987). For histochemical GUS assays, tissues were incubated in buffer containing 100 mM sodium phosphate (pH 7.0), 50 mM ascorbate and 1 mM 5-bromo-4-chloro-3-indolyl-β-D-glucuronide (X-gluc). Samples were incubated for up to 12 h at 37° C. and cleared with acetic acid:ethanol (1:3). For fluorometric GUS assays, total protein was extracted from leaf tissue and GUS activity assayed spectrophotometrically using 4-methylumbelliferone as the substrate.

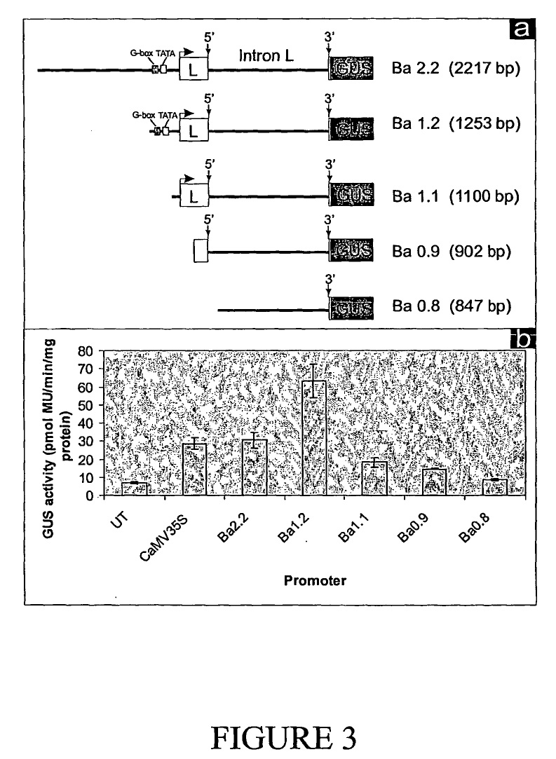

Thus, in order to determine regions of the ACT1 promoter responsible for activity, various truncations of the promoter were fused to a uidA reporter gene (FIG. 3a). The promoter fragments included the full length 2217 bp fragment (Ba2.2), a 1253 bp fragment containing the putative TATA and G-boxes (Ba1.2), a 1100 bp fragment which lacked these putative elements but still contained the complete 5′ UTL (Ba1.1), a 902 bp fragment including the 5′ intron splice site and part of the 5′ UTL (Ba0.9) and an 847 bp fragment lacking the 5′ intron splice site (Ba0.8). Varying levels of transient β-glucuronidase (GUS) activity were detected from all promoter fusions following microprojectile bombardment into banana embryogenic cell lines. The Ba1.2 promoter showed greatest activity and directed approximately 2-fold greater expression than the Ba2.2 and CaMV 35S promoters (FIG. 3b), but was 4-fold less active than the maize ubil promoter (results not shown). Removal of both the putative TATA and G-boxes (Ba1.1) resulted in a 5-fold decrease in expression relative to the Ba2.2 fragment, suggesting that these, or other, upstream elements are indeed important to promoter activity. Interestingly, removal of the transcriptional start site (Ba0.9) did not result in any significant decrease in expression, whilst a truncation removing the 5′ intron splice site (Ba0.8) reduced expression to background levels. Similar results have been reported by Dugdale et al., (2000) who demonstrated that a small part of the 5′ UTL and intron of both the maize ubil and rice ACT1 promoters were capable of directing significant GUS expression in banana. However, the origin and mechanism of transcription initiation from these minimal “promoter” fragments remains undetermined.

### Example 7

Banana Transformation and Regeneration

Ba-uidA promoter fusions were introduced into banana (Musa spp. cv. Bluggoe) embyrogenic cell suspensions using a particle inflow gun. Target tissue was prepared essentially as described by Dugdale et al., (1998). Preparation of microcarrier gold particles and coating of plasmid DNA were essentially as described by Becker et al., (2000). For transient expression studies, tissues were assayed for GUS activity 48-h post-bombardment. Transformed banana plants were selected and regenerated essentially as described by Becker et al., (2000).

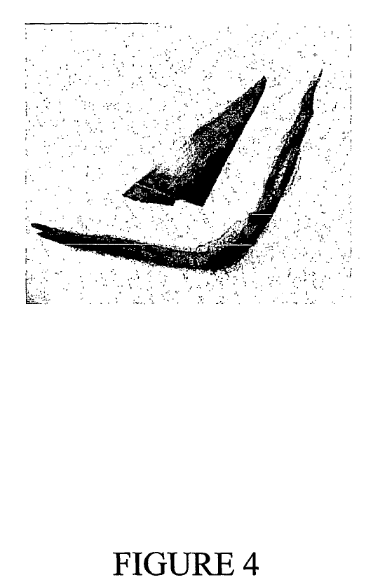

Thus, to study spatial and temporal regulation of the ACT1 promoter in whole plants, several independent banana lines transformed with either the Ba.2.2-uidA (three lines) or Ba1.2-uidA fusions (two lines) were regenerated and analysed. Histochemical GUS staining of both the Ba2.2-uidA and Ba1.2-uidA transformed plants revealed strong reporter expression throughout the pseudostem, leaves and roots (FIG. 4) suggesting a constitutive pattern of expression. Similar to transient assays, fluorometric assays from leaf extracts of transgenic plants indicated that the Ba1.2 promoter directed approximately two-fold greater expression than the Ba2.2 and CaMV 35S promoters, and 2-fold lower than the maize ubil promoter (Table 1). These results supported the northern hybridisation and ACT1 gene sequence analysis and suggested that the banana ACT1 gene belongs to the vegetative class of plant actins.

The study described herein has indicated that the banana ACT1 promoter appears to promote strong near-constitutive expression in banana. Of the few promoters assessed for activity in transgenic banana, only a few have demonstrated strong constitutive expression, namely maize ubil, CaMV 35S (Dugdale et al., 1998), the genomic promoter from sugarcane bacilliform badnavirus (Schenk et al., 1999) and at least one promoter from the banana bunchy top virus (Dugdale et al., 1998). Thus, the ACT1 promoter, which represents the first actin promoter characterised from a non-graminaceous monocot, may be useful for controlling transgene expression in banana and potentially in other plants including dicots and other monocots.

### Example 8

Transient Assay for Banana Actin Promoter Activity in Taro (Colocasia Esculenta)

Leaves were excised from in vitro taro plantlets and placed in petri dishes on agar solidified MS (Murashige and Skoog, 1962) medium abaxial side up. Leaves were transformed by microparticle bombardment. Constructs used employed GUS driven by either CaMV 35S or the Ba1.2 promoter fragment. Leaves were histochemically GUS stained two days post bombardment and the number of blue foci counted. The number of blue foci within an area of 28 mm² was counted in randomly chosen regions on three different leaves for each of the constructs. Values presented in Table 2 represent means of three replicates.

### Example 9

Tobacco Transformation I

Agrobacterium—Mediated Transformation and Regeneration

Tobacco (eg. N. tabacum: cv. Xanthi) plants are grown on MS media (Murashige and Skoog 1962) and subcultured monthly. Purified binary vectors are used to transform Agrobacterium tumefaciens LBA4404 by electroporation (Singh et al. 1993). The Agrobacterium transformants are then inoculated into LB medium containing kanamycin (100 mg/L) and grown overnight at 28° C. This culture is used to transform tobacco leaf discs as described by Horsch et al. (1988). Stably transformed tobacco plants are selected by the inclusion of kanamycin (100 mg/L) in the media. Timentin (200 mg/L) is also included to kill any residual Agrobacterium. Plantlets are regenerated as described by Horsch et al. (1988).

### Example 10

Tobacco Transformation II

Microprojectile Bombardment and Regeneration

The constructs are introduced into tobacco callus using a particle inflow gun. Target tissue is prepared essentially as described by Dugdale et al. (1998). Preparation of microcarrier gold particles and coating of plasmid DNA are essentially as described by Becker et al. (2000). For transient expression studies, tissues are usually assayed 48 h post-bombardment.

### Example 11

Banana Transformation and Regeneration

Constructs are introduced into banana (Musa spp) embyrogenic cell suspensions using a particle inflow gun. Target tissue is prepared essentially as described by Dugdale et al. (1998). Preparation of microcarrier gold particles and coating of plasmid DNA are essentially as described by Becker et al. (2000). For transient expression studies, tissues are usually assayed for reporter gene activity 48 h post-bombardment. Transformed banana plants are selected and regenerated essentially as described by Becker et al. (2000).

### Example 12

Sugarcane Transformation and Regeneration

Embryogenic callus of the Saccharum spp. (e.g., cv. Q117) is initiated from transverse sections of young leaf and sheath tissue and maintained on MSC₃ medium (Heinz & Mee, 1969) as described by Franks and Birch (1991). Preparation of callus for microprojectile bombardment is essentially by the method of Bower et al. (1996). Briefly, pieces of callus tissue of approximately 2 mm in diameter are subcultured onto fresh MSC₃ medium four days prior to microprojectile bombardment. Four hours prior to bombardment this callus is then transferred to MSC₃ containing 0.2 M sorbitol and 0.2 M mannitol. The callus is maintained on this medium for 3 hours post bombardment.

Callus is co-transformed by bombardment with tungsten microprojectiles using a particle inflow gun essentially as described in Bower et al. (1996). The microprojectiles are coated with 5 μg of the transformation construct together with 5 μg of the selectable marker plasmid pEmuKN. The target tissue is placed on a platform 7.5 cm from the point of discharge of the microprojectiles and covered with a protective screen of 210 μM stainless steel mesh. The particles are discharged with a helium pressure of 550 kPa in a chamber evacuated to 600 mm Hg. Three hours post bombardment all callus is then transferred to selection media containing 50 μg/mL geneticin.

Selection of transformed callus is based on visible GFP expression and by inclusion of 50 g/1 mL geneticin in the MSC₃ medium. Culture conditions and regeneration of transformed plants is exactly as described in Bower et al. (1996). Regenerated plantlets with visible GFP expression are then acclimatised in the glasshouse.

TABLES

TABLE 1
Mean fluorometric GUS activities (pmol MU/min/mg protein) from
leaf tissue of transgenic banana plants ± the standard error of the
mean (SEM). The number of lines of each plant is shown. The CaMV
35S and maize polyubiquitin (ubi1) promoters were included for
promoter comparisons and untransformed (UT) banana was included
as a background control.

| Promoter | Plant lines | Activity (pmol MU/min/mg protein) ± SEM |
|---|---|---|
| UT | 3 | 2.5 ± 0.2 |
| CaMV 35S | 2 | 1670 ± 1181 |
| UBI1 | 3 | 5338 ± 1771 |
| Ba2.2 | 3 | 1263 ± 510 |
| Ba1.2 | 2 | 3869 ± 871 |

TABLE 2

| Promoter construct | Number of blue foci (dots) |
|---|---|
| Control | 0 |
| CaMV 35S | 86 |
| Ba1.2 | 45 |

### BIBLIOGRAPHY

Altschul S F, Madden T L, Schaffer A A, Zhang J, Zhang A, Miller W, Lipman DJ (1997) Gapped BLAST and PSI-BLAST: a new generation of protein database search programs. Nucl Acids Res 25: 3389-3402.
An SS, Mopps B, Weber K, Bhattacharya D (1999) The origin and evolution of green algal and plant actins. Mol Biol Evol 16: 275-285.
An Y-Q, Huang S, McDowell J M, Mckinney E C, Meagher RB (1996a) The Arabidopsis ACT1/ACT3 actin subclass is expressed in organ primordia and mature pollen. Plant Cell 8: 15-30.
An Y-Q, McDowell J M, Huang S, McKinney E C, Chambliss S, Meagher R B (1996b) Strong, constitutive expression of the Arabidopsis ACT2/ACT8 actin subclass in vegetative tissues. Plant J10: 107-121.
Becker D K, Dugdale B, Smith M K, Harding R M, Dale J L (2000) Genetic transformation of Cavendish banana (Musa spp. AAA group) cv ‘Grand Nain’ via microprojectile bombardment. Plant Cell Rep 19: 229-234.
Bower, R., Elliott, A. R., Potier, B. A. M., Birch, R. G. (1996) High-efficiency, microprojectile-mediated cotransformation of sugarcane, using visible or selectable markers. Molecular Breeding 2: 239-249
Chang S, Puryear J, Caimey J (1993) A simple and efficient method for isolating RNA from pine trees. Plant Mol Biol Rep 11: 113-116.
Chibbar R, Kartha K, Datla R, Leung N, Caswell K, Mallard C, Steinhauer L (1993) The effect of different promoter-sequences on transient expression of gus reporter gene in cultured barley (Hordeum vulgare L.) cells. Plant Cell Rep 12: 506-509.
Christensen A H, Quail P H (1996) Ubiquitin promoter-based vectors for high-level expression of selectable and/or screenable marker genes in monocotyledonous plants. Transgenic Res 5: 213-218.
Dugdale B, Beetham PR, Becker D K, Harding R M, Dale J L (1998) Promoter activity associated with the intergenic regions of banana bunchy top virus DNA-1 to −6 in transgenic tobacco and banana cells. J Gen Virol 79: 2301-2311.
Dugdale B, Becker DK, Harding RM, Dale J L (2000) Intron-mediated enhancement of the banana bunchy top virus DNA-6 promoter in banana (Musa spp.) embryogenic cells and plants. Plant Cell Rep 20(3): 220-226.
Franks, T., Birch, R. G. (1991) Gene transfer into intact sugarcane cells using microprojectile bombardment. Australian Journal of Plant Physiology 18: 471-480
Frohman M A, Dush M K, Martin G R (1988) Rapid production of full-length cDNAs from rare transcripts: Amplification using a single gene-specific oligonucleotide primer. Proc Natl Acad Sci USA 85: 8998-9002.
Heinz, D. J, Mee, G. W. P. (1969) Plant differentiation from callus tissue of Saccharum species. Crop Science 9: 346-348.
Hightower R C, Meagher R B (1985) Divergence and differential expression of soybean actin genes. EMBO J 4:1-8.
Horsch R B, Fry J, Hoffmann N, Neidermeyer J, Rogers S G, Fraley RT (1988) Leaf disc transformation. In: Gelvin S B, Schilperoort R A (eds) Plant Molecular Biology Manual. Kluwer Academic Publishers, Dordecht, pp A5/1-A5/9.
Huang S, An Y-Q, McDowell J M, McKinney E C, Meagher R B (1996) The Arabidopsis thaliana ACT4/ACT12 actin gene subclass is strongly expressed throughout pollen development. Plant J 10: 189-202.
Huang S, An Y-Q, McDowell J M, McKinney E C, Meagher R B (1997) The Arabidopsis ACT1 actin gene is strongly expressed in tissues of the emerging inflorescence, pollen and developing ovules. Plant Mol Biol 33: 125-139.
Jefferson R A, Kavanagh T A, Bevan M W (1987) GUS fusions: β-glucuronidase as a sensitive and versatile gene fusion marker in higher plants. EMBO J6:3901-3907
Kandasamy M K, McKinney E C, Meagher R B (1999) The late pollen-specific actins in angiosperms. Plant J18: 681-691.
McDowell J M, Huang S, McKinney E C, An Y-Q, Meagher R B (1996a) Arabidopsis thaliana contains ten actin genes encoding six ancient protein subclasses. Genetics 142: 587-602.
McDowell J M, An Y-Q, Huang S, McKinney E C, Meagher R B (1996b) The Arabidopsis ACT7 actin gene is expressed in rapidly developing tissues and responds to several external stimuli. Plant Phys 111: 699-711.
McElroy D, Rothenberg M, Reece K S, Wu R (1990a) Characterisation of the rice (Oryza sativa) actin gene family. Plant Mol Biol 15: 257-268.
McElroy D, Zhang W, Cao J, Wu R (1990b) Isolation of an efficient actin promoter for use in rice transformation. Plant Cell 2: 163-171.
McElroy D, Rothenberg M, Wu R (1990c) Structural characterisation of a rice actin gene. Plant Mol Biol 14: 163-171.
McKinney E C, Meagher R B (1998) The Arabidopsis actin gene family members are widely dispersed in the genome. Genetics 149: 663-675.
Meagher R B, McKinney E C, Vitale A V (1999) The evolution of new structures: clues from plant cytoskeletal genes. Trends Genet 15: 278-284.
Muller P R, Wold B (1989) In vivo footprinting of a muscle specific enhancer by ligation mediated PCR. Science 246: 780-786.
Murashige T, Skoog F (1962) A revised medium for rapid growth and bioassays with tobacco tissue cultures. Physiol Plant 15: 473-497.
Rombauts S. Dehais P, Van Montagu M, Rouze p (1999) PlantC-RE, a plant cis-acting regulatory element database. Nucl Acids Res 27: 295-296.
Sambrook J, Fritsch E F, Maniatis T (1989) Molecular cloning, a laboratory manual. 2^{nd} edition, Vols. 1-3. Cold Spring Harbor Laboratory Press, Cold Spring Harbor, N.Y.
Schenk P M, Sagi L S, Remans T, Dietzgen R G, Bernard M J, Graham M W, Manners J M (1999) A promoter from sugarcane bacilliform badnavirus drives transgene expression in banana and other monocot and dicot plants. Plant Mol Biol 39: 1221-1230.
Simpson G G, Filipowicz W (1996) Splicing of precursors to mRNA in higher plants: mechanism, regulation and sub-nuclear organisation of the spliceosomal machinery. Plant Mol Biol 32: 1-41.
Singh A, Kao T H, Lin J J (1993) Transformation of Agrobacterium tumefaciens with T-DNA vector using high-voltage electroporation. Focus 15: 84-87.
Stewart C N, Via L E (1993) A rapid CTAB DNA isolation technique for RAPD fingerprint and PCR applications. Biotechniques 14: 748-750.
Takumi S, Otani M, Shimada T (1994) Effect of six promoter-intron combinations on transient reporter gene expression in einkorn, emmer and common wheat cells by particle bombardment. Plant Science 103: 161-66.
Thangavelu M, Belostotsky D, Bevan M, Flavell R, Rogers H, Lonsdale D (1993) Partial characterisation of the Nicotiana tabacum actin gene family: Evidence for pollen specific expression of one of the gene family members. Mol Gen Genet 240: 290-295.
Wilmink A, van de Ven B, Dons J (1995) activity of constitutive promoters in various species from the Liliaceae. Plant Mol Biol 28: 949-955.
Wingender E, Dietze P, Karas H, Knuppel R (1996) TRANSFAC: A database on transcription factors and their binding sites. Nucl Acids Res 24: 238-241.
Zhong H, Zhang S B, Warkentin D, Sun B L, Wu T Y, Wu R, Sticklen M B (1996) Analysis of the functional activity of the 1.40 KB 5′-region of the rice actin 1 gene in stable transgenic plants of maize (Zea mays L). Plant Science 116: 73-84.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-57. (canceled)

58. An isolated DNA molecule comprising a promoter or a biologically active fragment thereof or a variant of these, wherein the promoter comprises the sequence set forth in SEQ ID NO: 3.

59. The DNA molecule of claim 58, wherein the biologically active fragment is selected from the group consisting of SEQ ID NO: 4, 5, 6 and 7.

60. The DNA molecule of claim 58, wherein the variant has at least 60% sequence identity to a sequence selected from the group consisting of SEQ ID NO: 3, 4, 5, 6 and 7.

61. The DNA molecule of claim 58, wherein the variant is capable of hybridising to a sequence selected from the group consisting of SEQ ID NO: 3, 4, 5, 6 and 7 under at least low stringency conditions.

62. A chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO:3.

63. The construct of claim 62, further comprising a 3′ non-translated sequence that is operably linked to the foreign or endogenous DNA sequence and that functions in plant cells to terminate transcription and/or to cause addition of a polyadenylated nucleotide sequence to the 3′ end of a transcribed RNA sequence.

64. The construct of claim 62, wherein the biologically active fragment is selected from the group consisting of SEQ ID NO: 4, 5, 6 and 7.

65. The construct of claim 62, wherein the variant has at least 60% sequence identity to a sequence selected from the group consisting of SEQ ID NO: 3, 4, 5, 6 and 7.

66. The construct of claim 62, wherein the variant is capable of hybridising to a sequence selected from the group consisting of SEQ ID NO: 3, 4, 5, 6 and 7 under at least low stringency conditions.

67. The construct of claim 62, wherein the foreign or endogenous DNA sequence encodes a structural or regulatory protein.

68. The construct of claim 62, wherein the foreign or endogenous DNA sequence encodes a transcript capable of modulating expression of a corresponding target gene.

69. The construct of claim 68, wherein the transcript comprises an antisense RNA or a ribozyme or other transcribed region aimed at downregulation of expression of the corresponding target gene.

70. The construct of claim 62, further comprising an enhancer element.

71. The construct of claim 62, further comprising a leader sequence which modulates mRNA stability.

72. The construct of claim 62, further comprising a targeting sequence for targeting a protein product of the foreign or endogenous DNA sequence to an intracellular compartment within plant cells or to an extracellular environment.

73. The construct of claim 62, further comprising a selectable marker gene.

74. The construct of claim 62, further comprising a screenable marker gene.

75. The construct of claim 62, wherein the promoter, or biologically active fragment or variant is a constitutively active promoter.

76. The construct of claim 62, wherein the promoter, or biologically active fragment or variant is operable in a plant host cell.

77. The construct of claim 76, wherein the host cell is a monocotyledonous plant cell.

78. The construct of claim 76, wherein the host cell is a non-graminaceous monocotyledonous plant cell.

79. The construct of claim 76, wherein the host cell is a non-graminaceous monocotyledonous plant cell selected from the group consisting of Musaceae, taro, ginger, onions, garlic, pineapple, bromeliaeds, palms, orchids, lilies and irises.

80. The construct of claim 76, wherein the host cell is a graminaceous monocotyledonous plant cell.

81. The construct of claim 76, wherein the host cell is a dicotyledonous plant cell.

82. A method for gene expression in a plant, comprising introducing into a plant cell a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3.

83. A method for producing transformed plant cells, comprising:
(a) introducing into regenerable plant cells a chimeric DNA construct comprising a promoter or a biologically active fragment thereof or a variant of these, which is operably linked to a foreign or endogenous DNA sequence to be transcribed, wherein the promoter comprises the sequence set forth n SEQ ID NO: 3, so as to yield transformed plant cells; and
(b) identifying or selecting transformed plant cells.

84. A method for selecting stable genetic transformants from transformed plant cells comprising:
(a) introducing into regenerable plant cells a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3, so as to yield transformed plant cells; and
(b) identifying or selecting a transformed plant cell line from said transformed plant cells.

85. A method for producing a differentiated transgenic plant, comprising:
(a) introducing into regenerable plant cells a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3, so as to yield regenerable transformed plant cells;
(b) identifying or selecting a population of transformed plant cells; and
(c) regenerating a differentiated transgenic plant from said population.

86. The method of any one of claims 82 to 85, wherein the cell or cells are selected from dicotyledonous plant cells.

87. The method of any one of claims 82 to 85, wherein the cell or cells are selected from monocotyledonous plant cells.

88. The method of any one of claims 82 to 85, wherein the cell or cells are selected from graminaceous monocotyledonous plant cells.

89. The method of any one of claims 82 to 85, wherein the cell or cells are selected from non-graminaceous monocotyledonous plant cells.

90. The method of any one of claims 83 to 85, wherein expression of the chimeric DNA construct in the transformed cells imparts a phenotypic characteristic to the transformed cells.

91. The method of any one of claim 82 to 85, wherein the construct comprises a selectable marker gene.

92. The method of any one of claim 82 to 85, wherein the construct comprises a screenable marker gene.

93. The method of claim 85, wherein expression of the chimeric DNA construct renders the differentiated transgenic plant identifiable over the corresponding non-transgenic plant.

94. The method of claim 85, further comprising obtaining progeny from the differentiated transgenic plant.

95. Progeny obtained by the method of claim 94.

96. A plant part of the differentiated transgenic plant obtained by the method of claim 85, wherein the plant part contains the chimeric construct.

97. A differentiated transgenic plant regenerated from transformed plant cells obtained by the method of claim 83.

98. A transformed plant cell containing a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3.

99. A differentiated transgenic plant comprising plant cells containing a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3.

100. The transgenic plant of claim 99, wherein the plant is a dicotyledonous plant.

101. The transgenic plant of claim 99, wherein the plant is a monocotyledonous plant.

102. The transgenic plant of claim 99, wherein the plant is a graminaceous monocotyledonous plant.

103. The transgenic plant of claim 99, wherein the plant is a non-graminaceous monocotyledonous plant.

104. The transgenic plant of claim 99, wherein the construct comprises a selectable marker gene.

105. The transgenic plant of claim 99, wherein the construct comprises a screenable marker gene.

106. The transgenic plant of claim 99, wherein the expression of the chimeric DNA construct renders the differentiated transgenic plant identifiable over the corresponding non-transgenic plant.

107. Use of a chimeric DNA construct comprising a promoter or a biologically active fragment thereof or a variant of these, which is operably linked to a foreign or endogenous DNA sequence to be transcribed, wherein the promoter comprises the sequence set forth in SEQ ID NO: 3 in the production of a transformed plant cell, plant or plant part.


SEQUENCE LISTING (33 sequences)

SEQ ID NO: 1 - DNA, 1610 bp, Musa sp.
  5′UTR (1)..(11): Part of exon 1
atatccataacatggctgacggcgaggatatccagcctcttgtctgcgacaatggtactggaatggtcaaggttagacatccttttcctctcttatgttataatttgtccctctttccctgatcatgttgcaaacaacatatataactgttctctttcaggctggtttcgccggagatgatgcacctagagctgtattccctagcatcgtaggccgacctcgccacactggtgtcatggttgggatgggtcaaaaagatgcttatgttggtgacgaggctcaatcaaagagaggtatcctgaccttgaagtaccctattgaacacggcattgttagcaactgggatgacatggagaagatctggcatcacaccttctataatgagcttcgtgttgcccctgaggagcaccctgtattgctcaccgaagcccctctcaatcccaaggcaaaccgagagaagatgacccagattatgtttgaaaccttcaatgttccggctatgtatgtagcaattcaggctgttctttccctttatgctagtggtcgtacaaccggtatgcctcacttcaacttgattaaagtctcaaacatgacatgtaaatgctgatctgtggcactcttcttaataggtattgtgcttgattctggtgatggtgtgagccacactgttcctatatacgaaggatatgccctccctcatgccatccttcgtttggaccttgctggtcgtgacctcacagatgctttgatgaagattcttacggagagaggttattccttcactaccaccgctgaacgggaaattgtcagggatatcaaggaaaagcttgcctacgttgccctggattatgaacaggagctggagaatgccaagagtagttcctctgtggaaaagagctatgagttacctgatgggcaggtcatcacaattggtgcggagagattcagatgcccggaggttctcttccagccatctctgatcggcatggaggctgctggtatccatgagaccacttacaattccatcatgaagtgtgatgtggatatcagaaaggacctgtatggcaacatcgttctcagtggtggtacgactatgtttcctggaatcgctgaccgcatgagcaaggagatcacagcgctcgccccaagcagcatgaagattaaggtggttgccccaccagagcgcaagtacagtgtctggattggaggctccatccttgcttcgctcagcacttttcagcaggtaatatcttgatccacatttgcaaattatcaagattcagaaacgtttcgcagctatttcatgatcataatttctttctctttttttgtttaataatgcaccagatgtggatctccaagggcgaatacgaggaatccggtccttccattgtccacaggaagtgcttctgagttccatggtccccaagtcacggttactactcttcaggctgcttttcaagactgaatcacgcgagtctttttccaaattgatgtggttttgttggattcgctcgagttgtacctgtactttaggtaccataaatatatgtgaactgccataagatcactgcttaaaatctgtttctgcaatgaaggctgtttatcga

SEQ ID NO: 2 - protein, 377 aa, Musa sp.
MADGEDIQPLVCDNGTGMVKAGFAGDDAPRAVFPSIVGRPRHTGVMVGMGQKDAYVGDEAQSKRGILTLKYPIEHGIVSNWDDMEKIWHHTFYNELRVAPEEHPVLLTEAPLNPKANREKMTQIMFETFNVPAMYVAIQAVLSLYASGRTTGIVLDSGDGVSHTVPIYEGYALPHAILRLDLAGRDLTDALMKILTERGYSFTTTAEREIVRDIKEKLAYVALDYEQELENAKSSSSVEKSYELPDGQVITIGAERFRCPEVLFQPSLIGMEAAGIHETTYNSIMKCDVDIRKDLYGNIVLSGGTTMFPGIADRMSKEITALAPSSMKIKVVAPPERKYSVWIGGSILASLSTFQQMWISKGEYEESGPSIVHRKCF

SEQ ID NO: 3 - DNA, 2217 bp, Musa sp.
  promoter (1)..(2217)
  misc_feature (1076)..(1081): G-box like motif
accttagtctgagagctctgatgcactagatcgtaaacccactcttaggattatccgagaatacccaccctacaaatcgagcctacctttccacacttacatcacatatctaccgccgaggctgatcttttctcttattaataaggtgctgaaagcccactaccgacaagaactcaataagtacaagcaaggcagcaaccagtgcttccaatacggatagaggaagttcagactcaagccggccagttgcagcctgcagctcaggtacccttagactcaaaccgggcagctcgagccttgaactcaccaattacagactgaaaccgggcagctcaagccttggatcgatcggtcaaagactaaaacgagcttccccaaggccctaacccggcagcaacagaattaacccaatgagtataagaattgaaccggtaagtataaggctagttccagtccaaaagtaagaattgcactaatgggtctcaggcttagagctagtggcaacagaatggagccggtaaatcaaagcctgtagtggtaggaactcggacaaagactcagaattacacctatgagtcgaagccaacaagcagacaagccgacaagacacggatagacaagcagacaaaccacactttagtcaagtactctttcgacatttgcatcgagccaagtcactactaatgtgacgcggcagcgttgttgcacgggaaaacggaaaaaatcttagaaatctggtcagctattaggggaaatctggtcactgccaaaaaacccgggttttgtcaagaaatcctcgaaattgacccagctattagcggaaatccggttgctaccaaaaaacacgggttttggtggacgcccaccccgcctcaaccttccaacaccgcacgtcatgccggacctgatccgacggcctagatgaccctgatccgagctccatcggacgattggcgttccctctcgagacagctcattgacgaacacaaaaggcatattcgacttccgtgtatagctcaagcgacaaaacactgggtttaatctttttattcgacattctctgtgggatccgcaacacgtatcctaaatcccataagtcgagttaatagaaagaccatttaagctgtctcgtcgctgggtagagacacaggagctgcttgttcttttcctttttctctctgtttatctttgatctcgctactgcaatgatacccgcaaaattttcgtgacagaaatttagcgaaatccattcgatgtcttcctatttaatgcccttccactcgcctcgctccccgtctctctcgtcgccgctctgcacctctcactctccatctctcggggaagcgcagaggggcttcggtgtaaggtcagtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 4 - DNA, 1253 bp, Musa sp.
cgagacagctcattgacgaacacaaaaggcatattcgacttccgtgtatagctcaagcgacaaaacactgggtttaatctttttattcgacattctctgtgggatccgcaacacgtatcctaaatcccataagtcgagttaatagaaagaccatttaagctgtctcgtcgctgggtagagacacaggagctgcttgttcttttcctttttctctctgtttatctttgatctcgctactgcaatgatacccgcaaaattttcgtgacagaaatttagcgaaatccattcgatgtcttcctatttaatgcccttccactcgcctcgctccccgtctctctcgtcgccgctctgcacctctcactctccatctctcggggaagcgcagaggggcttcggtgtaaggtcagtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 5 - DNA, 1100 bp, Musa sp.
tttaagctgtctcgtcgctgggtagagacacaggagctgcttgttcttttcctttttctctctgtttatctttgatctcgctactgcaatgatacccgcaaaattttcgtgacagaaatttagcgaaatccattcgatgtcttcctatttaatgcccttccactcgcctcgctccccgtctctctcgtcgccgctctgcacctctcactctccatctctcggggaagcgcagaggggcttcggtgtaaggtcagtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 6 - DNA, 902 bp, Musa sp.
cacctctcactctccatctctcggggaagcgcagaggggcttcggtgtaaggtcagtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 7 - DNA, 847 bp, Musa sp.
gtcgacttcgtcttccgatctgtgttaagcatgtagaatgccatcgattttggtttattttttaggttttcgcgagttctcttgtaggatctacgttgtattagatgcgtataaattgttctattaactggttcagaaaatgattgttgattcgtatcggtacatcgtggatcgagcgaagatttggtaacttattgaccttcggtgttcagcttctctgttcttgccttttctgcctcattatcttgtttcttgattctttaagcttgcgatgataagatattgtgtttggatattgtgtgaaggattgctcttcctttggtttctgattctggaacaatttgattctcccatatattgcttttatattgagttcctctattaatatctgtttgatgatatttttattcatgatggtcgatcttaaaatatgaaaattaccttatgttgattgttcatcagctgtgaagctgaaatttggaaaagaaaatattcaataactgtttttttaagggaaaatatcgaattattacacctcgaaatatgttagttatgaacaatatgaagtggtcaatgattttacctgattaattcttgtcacataatatcaatacttgctttgaaactttcgaaacagtttcgttgtagtttcttgctggacatagttgcagatttctcagttactcttttgagtgttaaaccagcctttcctgtgtttatggaaggattagtgtatataaatagaagattattttgatgttgaattattgggtattaatttgctagagcatgtgggtggaattatatgttcttcctccttcgatttggttttgcagatatccataac

SEQ ID NO: 8 - DNA, 26 bp, Artificial Sequence
  LINKsac primer
agaattctgcaggatcccggggagct

SEQ ID NO: 9 - DNA, 22 bp, Artificial Sequence
  KNIL primer
ccccgggatcctgcagaattcg

SEQ ID NO: 10 - DNA, 20 bp, Artificial Sequence
  Actin-B primer
accttgaccattccagtgcc

SEQ ID NO: 11 - DNA, 18 bp, Artificial Sequence
  Actin-N primer
agtgccattgtcacagac

SEQ ID NO: 12 - DNA, 23 bp, Artificial Sequence
  BACT1-B
gatacgtgttgcggatcccacag

SEQ ID NO: 13 - DNA, 18 bp, Artificial Sequence
  BACT1-N primer
acggaagtcgaatatgcc

SEQ ID NO: 14 - DNA, 21 bp, Artificial Sequence
  BACT15.0 primer
accttagtctgagagctctga

SEQ ID NO: 15 - DNA, 21 bp, Artificial Sequence
  BACT13.0 primer
gttatggatatctgcaaaacc

SEQ ID NO: 16 - DNA, 27 bp, Artificial Sequence
  6-FAM end-labelled primer
gtcagccatgttatggatatcttacac

SEQ ID NO: 17 - DNA, 22 bp, Artificial Sequence
  Ba5utrF primer
cacctctcactctccatctctc

SEQ ID NO: 18 - DNA, 20 bp, Artificial Sequence
  Actex3r primer
cacttcatgatggagttgta

SEQ ID NO: 19 - DNA, 18 bp, Artificial Sequence
  ACT1 promoter specific primer BaIF
gttcttcctccttcgatt

SEQ ID NO: 20 - DNA, 17 bp, Artificial Sequence
  Actin primer
tagaagcacttcatgtg

SEQ ID NO: 21 - DNA, 18 bp, Artificial Sequence
  Actex2f primer
ccctgaggagcaccctgt

SEQ ID NO: 22 - DNA, 10 bp, Musa sp.
ttttgcagat

SEQ ID NO: 23 - DNA, 10 bp, Artificial Sequence
  Monocot consensus 3′ intron slice site
ttttgcaggt

SEQ ID NO: 24 - DNA, 8 bp, Musa sp.
aggtcagt

SEQ ID NO: 25 - DNA, 8 bp, Artificial Sequence
  Monocot consensus 5′ intron splice site
aggtaagt

SEQ ID NO: 26 - DNA, 6 bp, Artificial Sequence
  G-box like motif
cacgta

SEQ ID NO: 27 - DNA, 6 bp, Artificial Sequence
  TATA-box like motif
ttaata

SEQ ID NO: 28 - DNA, 20 bp, Artificial Sequence
  Ba3utrF primer
caggaagtgcttctgagttc

SEQ ID NO: 29 - DNA, 20 bp, Artificial Sequence
  Ba3utrR primer
ataaacagccttcattgcag

SEQ ID NO: 30 - DNA, 18 bp, Artificial Sequence
  18Sf primer
cagactgtgaaactgcga

SEQ ID NO: 31 - DNA, 18 bp, Artificial Sequence
  18Sr primer
gctttcgcagtggttcgt

SEQ ID NO: 32 - DNA, 25 bp, Artificial Sequence
  Bact5.1 primer
cgagacagctcattgacgaacacaa

SEQ ID NO: 33 - DNA, 18 bp, Artificial Sequence
  Bact5.2 primer
tttaagctgtctcgtcgc